(19) United States
(12) Patent Application Publication
(10) Pub. No.: US 2015/0306236 A1
(43) Pub. Date: Oct. 29, 2015

(54) BOLAAMPHIPHILIC COMPOUNDS, COMPOSITIONS AND USES THEREOF

(71) Applicant: Lauren Sciences LLC, New York, NY (US)

(72) Inventors: Charles LINDER, Rehovot (IL); Eliahu HELDMAN, Rehovot (IL); Sarina GRINBERG, Meitar (IL)

(21) Appl. No.: 14/638,466

(22) Filed: Mar. 4, 2015

Related U.S. Application Data
(63) Continuation of application No. PCT/US2013/057960, filed on Sep. 4, 2013.
(60) Provisional application No. 61/696,798, filed on Sep. 4, 2012.
(60) Provisional application No. 61/974,201, filed on Apr. 2, 2014.

(51) Int. Cl. A61K 47/48; A61K 31/724; A61K 38/08; A61K 31/475; A61K 38/05

(52) U.S. Cl. CPC A61K 47/4883; A61K 31/475; A61K 38/05; A61K 38/08; A61K 31/724; A61K 47/482; A61K 47/48176; A61K 47/48923

(57) ABSTRACT

Bolaamphiphilic compounds are provided according to formula I:

HG²-L¹-HG¹ I

where HG¹, HG² and L¹ are as defined herein. Provided bolaamphilphilic compounds and the pharmaceutical compositions thereof are useful for delivering biologically active drugs into animal or human brain.

### CROSS REFERENCE TO RELATED APPLICATIONS

This application is a continuation of International Application No. PCT/US2013057960, filed Sep. 4, 2013, which claims priority to U.S. Application No. 61/696,798, filed Sep. 4, 2012, the contents of which are incorporated by reference herein. This application further claims priority to U.S. Application No. 61/974,201 filed Apr. 2, 2014, the contents of which are also incorporated by reference herein.

### FIELD

Provided herein are nanovesicles comprising bolaamphiphilic compounds, and complexes thereof with biologically active molecules, and pharmaceutical compositions thereof Also provided are methods of delivering biologically active molecules into the human brain and animal brain using the compounds, complexes and pharmaceutical compositions provided herein.

### BACKGROUND

Many drugs and biologically active molecules cannot penetrate the BBB and thus require direct administration into the CNS tissue or the cerebral spinal fluid (CSF) in order to achieve a biological or therapeutic effect. Even direct administration into a particular CNS site is often limited due to poor diffusion of the active agent because of local absorption/adsorption into the CNS matrix. Present modalities for drug delivery through the BBB entail disruption of the BBB by, for example, osmotic means (hyperosmotic solutions) or biochemical means (e.g., use of vasoactive substances such as. bradykinin), processes with serious side effects.

The brain is a highly specialized organ, and its sensitive components and functioning are protected by a barrier known as the blood-brain barrier (BBB). The brain capillary endothelial cells (BCECs) that form the BBB play important role in brain physiology by maintaining selective permeability and preventing passage of various compounds from the blood into the brain. One consequence of the highly effective barrier properties of the BBB is the limited penetration of therapeutic agents into the brain, which makes treatment of many brain diseases extremely challenging.

Efforts to improve the permeation of biologically active compounds across the BBB using amphphilic vesicles have been attempted.

For example, complexation of the anionic carboxyfluorescein (CF) with single headed amphiphiles of opposite charge in cationic vesicles, formed by mixing single-tailed cationic and anionic surfactants has been reported (Danoff et al. 2007).

Furthermore, WO 02055011 and WO 03047499, both of the same applicant, disclose amphiphilic derivatives composed of at least one fatty acid chain derived from natural vegetable oils such as vernonia oil, lesquerella oil and castor oil, in which functional groups such as epoxy, hydroxy and double bonds were modified into polar and ionic headgroups.

Additionally, WO 10128504 reports a series of amphiphiles and bolamphiphiles (amphiphiles with two head groups) useful for targeted drug delivery of insulin, insulin analogs, TNF, GDNF, DNA, RNA (including siRNA), enkephalin class of analgesics, and others.

These synthetic bolaamphiphiles (bolas) have recently been shown to form nanovesicles that interact with and encapsulate a variety of small and large molecules including peptides, proteins and plasmid DNAs delivering them across biological membranes. These bolaamphiphiles are a unique class of compounds that have two hydrophilic headgroups placed at each ends of a hydrophobic domain. Bolaamphiphiles can form vesicles that consist of monolayer membrane that surrounds an aqueous core. Vesicles made from natural bolaamphiphiles, such as those extracted from archaebacteria (archaesomes), are very stable and, therefore, might be employed for targeted drug delivery. However, bolaamphiphiles from archaebacteria are heterogeneous and cannot be easily extracted or chemically synthesized.

Thus, there remains a need to make new compositions and for novel methods to deliver biologically active drugs into the brain. The compounds, compositions, and methods described herein are directed toward this end.

### SUMMARY OF THE INVENTION

In certain aspects, provided herein are pharmaceutical compositions comprising of a bolaamphiphile complex.

In further aspects, provided herein are novel nano-sized vesicles comprising of bolaamphiphilic compounds.

In certain aspects, provided herein are novel bolaamphiphile complexes comprising one or more bolaamphiphilic compounds and a biologically active compound.

In one embodiment, the biologically active compound is a compound active against ALS. In another embodiment, the biologically active compound is an analgesic compound.

In further aspects, provided herein are novel formulations of biologically active compounds with one or more bolaamphiphilic compounds or with bolaamhphile vesicles.

In another aspect, provided here are methods of delivering biologically active drugs agents into animal or human brain. In one embodiment, the method comprises the step of administering to the animal or human a pharmaceutical composition comprising of a bolaamphiphile complex; and wherein the bolaamphiphile complex comprises one or more bolaamphiphilic compounds and a compound active against ALS. In one particular embodiment, the biologically active compound is an analgesic compound.

In one embodiment, the bolaamphiphilic compound consists of two hydrophilic headgroups linked through a long hydrophobic chain. In another embodiment, the hydrophilic headgroup is an amino containing group. In a specific embodiment, the hydrophilic headgroup is a tertiary or quaternary amino containing group.

In one particular embodiment, the bolaamphiphilic compound is a compound according to formula I:

HG²-L¹-HG¹ I

or a pharmaceutically acceptable salt, solvate, hydrate, prodrug, stereoisomer, tautomer, isotopic variant, or N-oxide thereof, or a combination thereof;
wherein:

each HG¹ and HG² is independently a hydrophilic head group; and

L¹ is alkylene, alkenyl, heteroalkylene, or heteroalkenyl linker; unsubstituted or substituted with C₁-C₂₀ alkyl, hydroxyl, or oxo.

In one embodiment, the pharmaceutically acceptable salt is a quaternary ammonium salt.

In one embodiment, with respect to the bolaamphiphilic compound of formula I, the bolaamphiphilic compound is a compound according to formula II, III, IV, V, or VI:

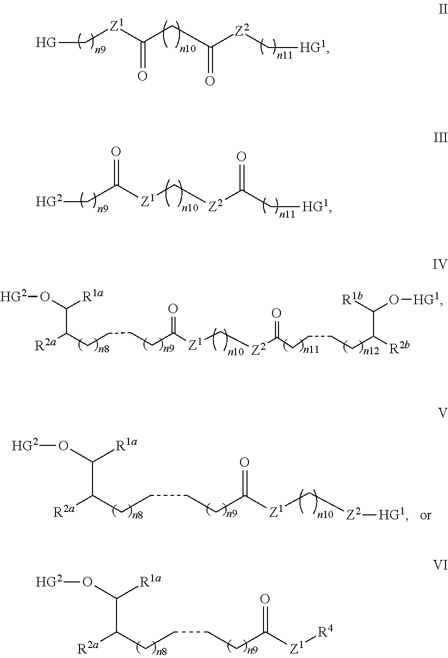

or a pharmaceutically acceptable salt, solvate, hydrate, prodrug, stereoisomer, tautomer, isotopic variant, or N-oxide thereof, or a combination thereof;
wherein:

each HG¹ and HG² is independently a hydrophilic head group;

each Z¹ and Z² is independently —C(R³)₂—, —N(R³)— or —O—;

each R^{1a}, R^{1b}, R³, and R⁴ is independently H or C₁-C₈ alkyl;

each R^{2a} and R^{2b} is independently H, C₁-C₈ alkyl, OH, alkoxy, or O—HG¹ or O—HG²;

each n8, n9, n11, and n12 is independently an integer from 1-20;

n10 is an integer from 2-20; and

each dotted bond is independently a single or a double bond.

In one embodiment, with respect to the bolaamphiphilic compound of formula I, II, III, IV, V, or VI, each HG¹ and HG² is independently selected from:

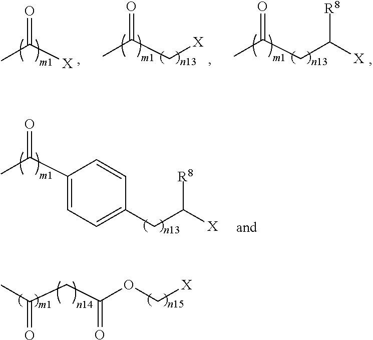

wherein:
X is —R^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
each R^{5c} is independently substituted or unsubstituted C₁-C₂₀ alkyl; each R⁸ is independently H, substituted or unsubstituted C₁-C₂₀ alkyl, alkoxy, or carboxy;
m1 is 0 or 1; and
each n13, n14, and n15 is independently an integer from 1-20.

In another embodiment, the present disclosure provides bolaamphiphiles, methods for the synthesis and use thereof, and compositons comprising same, that may be prepared from jojoba oil.

In another embodiment, the present disclosure provides bolaamphiphiles described within this application, methods for the synthesis and use thereof, and compositons comprising same, that include cyclodextrins within the compositions that form vesicles.

In another embodiment, the present disclosure provides bolaamphiphiles comprising specific targeting ligands, methods for the synthesis and use thereof, and compositons comprising same, that may used, e.g., for the treatment of brain tumors. In one aspect of this embodiment, the targeted brain tumor is a glioblastoma multiforme (GBM).

Other objects and advantages will become apparent to those skilled in the art from a consideration of the ensuing detailed description.

### FIGURES

FIG. 1A: TEM micrograph of vesicles from GLH-20 and their size distribution determined by DLS (FIG. 1B).

FIG. 2A: Head group hydrolysis by AChE of GLH-19 (blue) and GLH-20 (red) and release of CF from GLH-19 vesicles (FIG. 2B) and GLH-20 vesicles (FIG. 2C)

FIG. 3A: CF accumulation in brain after i.v. injection of encapsulated and non-encapsulated CF. Only GLH-20 vesicles allow accumulation of CF in the brain. CS improves GLH-20 vesicles' penetration into the brain (FIG. 3B).

FIG. 4A: Analgesia after i.v. injection of enkephalin non-encapsulated and encapsulated in vesicles. Analgesia (compared with morphine, which was used as a positive control) is obtained only when enkephalin is encapsulated in GLH-20 vesicles, the head groups of which are hydrolyzed by ChE. The vesicles do not disrupt the BBB since non-encapsulated enkephalin co-injected with empty vesicles (extravesicular enkephalin) did not cause analgesia (FIG. 4B). **Significantly different from free leu-enkephalin (t-test, P<0.01). ***Significantly different from free leu-enkephalin (t-test, P<0.001).

FIG. 5A: Fluorescence in mouse cerebral cortex after i.v. injection of albumin-FITC (non-encapsulated) encapsulated in GLH-20 vesicles (FIG. 5B).

FIG. 6: Brain delivery of analgesic peptide kyotorphin.

FIG. 7: ¹H-NMR and ¹³C-NMR of Compound (4)

FIG. 8A: MALDI spectrum of jojoba dichloroacetate; (FIG. 8B) Comparison between theoreticqal and actual distrution abundance of isotopes in C4₆H₈₆Cl₂O₆.

FIG. 9: ¹H-NMR and ¹³C-NMR of the bolaamphiphile GLH-58.

FIG. 10: MS (ESI) ([M-2Cl]⁺/2) of bolaamphiphile GLH-58.

FIG. 11: ¹H-NMR and ¹³C-NMR spectrua of the tetrachloroacetate of jojoba oil (10).

FIG. 12A: MALDI spectrum of tetracholoracetate of jojoba oil (compound (10)) and (FIG. 12B) of the bolaamphiphile GLH-60.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers andor diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

“Alkyl” refers to a radical of a straightchain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl.

“Alkylene” refers to a substituted or unsubstituted alkyl group, as defined above, wherein two hydrogens are removed to provide a divalent radical. Exemplary divalent alkylene groups include, but are not limited to, methylene (—CH₂—), ethylene (—CH₂CH₂—), the propylene isomers (e.g., —CH₂CH₂CH₂— and —CH(CH₃)CH₂—) and the like.

“Alkenyl” refers to a radical of a straightchain or branched hydrocarbon group having from 2 to 20 carbon atoms, one or more carboncarbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carboncarbon double bonds can be internal (such as in 2-butenyl) or terminal (such as in 1-butenyl). Examples of C₂ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers a substituted or unsubstituted alkenyl group, as defined above, wherein two hydrogens are removed to provide a divalent radical. Exemplary divalent alkenylene groups include, but are not limited to, ethenylene (—CH═CH—), propenylenes (e.g., —CH═CHCH₂— and —C(CH₃)═CH— and —CH═C(CH₃)—) and the like.

“Alkynyl” refers to a radical of a straightchain or branched hydrocarbon group having from 2 to 20 carbon atoms, one or more carboncarbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carboncarbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers a substituted or unsubstituted alkynyl group, as defined above, wherein two hydrogens are removed to provide a divalent radical. Exemplary divalent alkynylene groups include, but are not limited to, ethynylene, propynylene, and the like.

“Aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

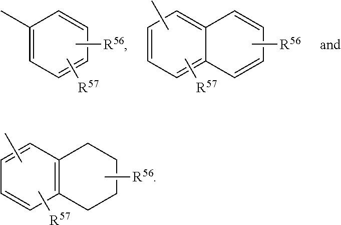

In these formulae one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbon in common with a second aryl ring or with an aliphatic ring.

“Aralkyl” is a subset of alkyl and aryl, as defined herein, and refers to an optionally substituted alkyl group substituted by an optionally substituted aryl group.

“Heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (arylheteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

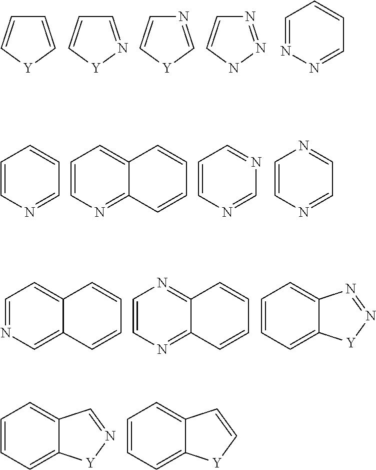

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

Examples of representative aryl having hetero atoms containing substitution include the following:

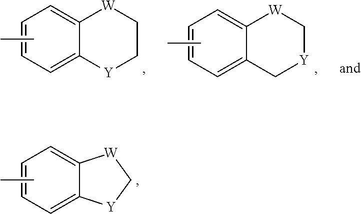

wherein each W is selected from C(R⁶⁶)₂, NR⁶⁶, O, and S; and each Y is selected from carbonyl, NR⁶⁶, O and S; and R⁶⁶ is independently hydrogen, C₁-C₈ alkyl, C₃-C₁₀ cycloalkyl, 4-10 membered heterocyclyl, C₆-C₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl” or “carbocyclic” refers to a radical of a nonaromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered nonaromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered nonaromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

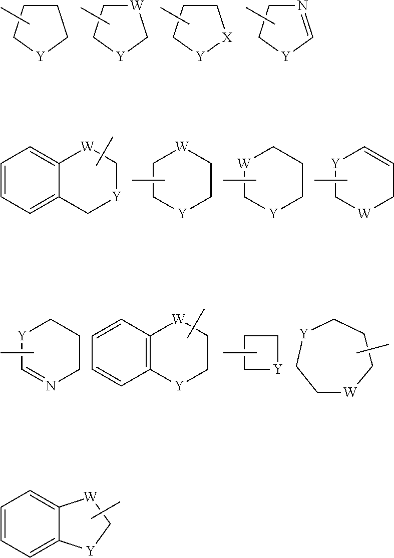

wherein each W is selected from CR⁶⁷, C(R⁶⁷)₂, NR⁶⁷, O, and S; and each Y is selected from NR⁶⁷, O, and S; and R⁶⁷ is independently hydrogen, C₁-C₈ alkyl, C₃-C₁₀ cycloalkyl, 4-10 membered heterocyclyl, C₆-C₁₀ aryl, 5-10 membered heteroaryl. These heterocyclyl rings may be optionally substituted with one or more substituents selected from the group consisting of the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g,. heteroaryl, cycloalkenyl, e.g., cycloheteroalkenyl, and the like having from 1 to 5, and particularly from 1 to 3 heteroatoms.

“Acyl” refers to a radical —C(O)R²⁰, where R²⁰ is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. “Alkanoyl” is an acyl group wherein R²⁰ is a group other than hydrogen. Representative acyl groups include, but are not limited to, formyl(—CHO), acetyl(—C(═O)CH₃), cyclohexylcarbonyl, cyclohexylmethylcarbonyl, benzoyl(—C(═O)Ph), benzylcarbonyl(—C(═O)CH₂Ph), —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Acylamino” refers to a radical —NR²²C(O)R²³, where each instance of R²² and R23 is independently hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl as defined herein, or R²² is an amino protecting group. Exemplary “acylamino” groups include, but are not limited to, formylamino, acetylamino, cyclohexylcarbonylamino, cyclohexylmethyl-carbonylamino, benzoylamino and benzylcarbonylamino. Particular exemplary “acylamino” groups are —NR²⁴C(O)—C₁-C₈ alkyl, —NR²⁴C(O)—(CH₂)ₜ(C₆-C₁₀ aryl), —NR²⁴C(O)—(CH₂)ₜ(5-10 membered heteroaryl), —NR²⁴C(O)—(CH₂)ₜ(C₃-C₁₀ cycloalkyl), and —NR²⁴C(O)—(CH₂)ₜ(4-10 membered heterocyclyl), wherein t is an integer from 0 to 4, and each R²⁴ independently represents H or C₁-C₈ alkyl. In certain embodiments, R²⁵ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; and R²⁶ is H, C₁-C₈ alkyl, substituted with halo or hydroxy;
C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C_{1—}C₄ haloalkoxy or hydroxyl; provided that at least one of R²⁵ and R²⁶ is other than H.

“Acyloxy” refers to a radical —OC(O)R²⁷, where R²⁷ is hydrogen, substituted or unsubstitued alkyl, substituted or unsubstitued alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl and benzylcarbonyl. In certain embodiments, R²⁸ is C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance, from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³⁸ is hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R³⁸ is not a hydrogen. In certain embodiments,each R³⁸ is independently selected from: hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary ‘substituted amino’ groups are —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Azido” refers to the radical —N₃.

“Carbamoyl” or “amido” refers to the radical —C(O)NH₂.

“Substituted carbamoyl” or “substituted amido” refers to the radical —C(O)N(R⁶²)₂ wherein each R⁶² is independently hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R⁶² is not a hydrogen. In certain embodiments, R⁶² is selected from H, C₁-C₈ alkyl, C₃-C₁₀ cycloalkyl, 4-10 membered heterocyclyl, C₆-C₁₀ aryl, aralkyl, 5-10 membered heteroaryl, and heteroaralkyl; or C₁-C₈ alkyl substituted with halo or hydroxy; or C₃-C₁₀ cycloalkyl, 4-10 membered heterocyclyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; provided that at least one R⁶² is other than H.

Exemplary ‘substituted carbamoyl’ groups include, but are not limited to, —C(O) NR⁶⁴—C₁-C₈ alkyl, —C(O)NR⁶⁴—(CH₂)ₜ(C₆-C₁₀ aryl), —C(O)N⁶⁴—(CH₂)ₜ(5-10 membered heteroaryl), —C(O)NR⁶⁴—(CH₂)ₜ(C₃-C₁₀ cycloalkyl), and —C(O)NR⁶⁴—(CH₂)ₜ(4-10 membered heterocyclyl), wherein t is an integer from 0 to 4, each R⁶⁴ independently represents H or C₁-C₈ alkyl and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Carboxy’ refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro. In further embodiments, the halo group is iodo.

“Hydroxy” refers to the radical —OH.

“Nitro” refers to the radical —NO₂.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Cycloalkenyl” refers to substituted or unsubstituted carbocyclyl group having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Fused cycloalkenyl” refers to a cycloalkenyl having two of its ring carbon atoms in common with a second aliphatic or aromatic ring and having its olefinic unsaturation located to impart aromaticity to the cycloalkenyl ring.

“Ethenyl” refers to substituted or unsubstituted —(C═C)—.

“Ethylene” refers to substituted or unsubstituted —(C—C)—.

“Ethynyl” refers to —(C≡C)—.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents andor any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(=O)R^{aa}, ═NNR^{bb}C(═O)OR^{aa}, ═NNR^{bb}S(═O)₂R^{aa}, ═NR^{bb}, or =NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂-₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{ee}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{ce})N(R^{ee})₂, —SO₂N(R^{cc})₂, —SO₂R^{cc}, —SO₂OR^{cc}, —SOR^{aa}, —C(═S)N(R^{ee})₂, —C(═O)SR^{ee}, —C(═S)SR^{ee}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-ditbutylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, mchloropacyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), mnitrophenyl carbamate, 3,5-dimethoxybenzyl carbamate, onitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl 4-methoxybenzenesulfonamide (Pme), 2,3,5,6-tetramethyl-4-methoxybenzenesulfonamide (Mte), 4-methoxybenzenesulfonamide (Mbs), 2,4,6trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxolcyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyllmethoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, tbutyl, allyl, pchlorophenyl, pmethoxyphenyl, 2,4-dinitrophenyl, benzyl (Bn), p-methoxybenzyl, 3,4-dimethoxybenzyl, onitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl-N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate(levulinate), 4,4-(ethylenedithio)pentanoate(levulinoyldithioacetal), pivaloate, adamantoate, crotonate, 4-methoxycrotonate, benzoate, p-phenylbenzoate, 2,4,6-trimethylbenzoate(mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl onitrobenzyl carbonate, alkyl pnitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is an sulfur protecting group (also referred to as a thiol protecting group). Sulfur protecting groups include, but are not limited to, —R^{aa}, —N(R^{bb})₂, —(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{ee})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

“Compounds of the present invention”, and equivalent expressions, are meant to embrace the compounds as hereinbefore described, in particular compounds according to any of the Formula herein recited andor described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like (see, e.g., Berge, et al., J. Pharm. Sci. 66(1): 1-79 (January '77).

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural Formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Prodrugs” refers to compounds, including derivatives of the compounds of the invention,which have cleavable groups and become by solvolysis or under physiological conditions the compounds of the invention that are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. Particularly the C₁ to C₈ alkyl, C₂-C₈ alkenyl, C₂-C₈ alkynyl, aryl, C₇-C₁₂ substituted aryl, and C₇-C₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or adult subject (e.g., young adult, middleaged adult or senior adult)) andor a non-human animal, e.g., a mammal such as primates (e.g., cynomolgus monkeys, rhesus monkeys), cattle, pigs, horses, sheep, goats, rodents, cats, andor dogs. In certain embodiments, the subject is a human. In certain embodiments, the subject is a non-human animal. The terms “human”, “patient” and “subject” are used interchangeably herein.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Preventing” or “prevention” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Treating” or “treatment” of any disease or disorder refers, in certain embodiments, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²HD, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug andor substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, which are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure S-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

### DETAILED DESCRIPTION OF THE PREFERRED EMBODIMENTS

In certain aspects, provided herein are pharmaceutical compositions comprising of a bolaamphiphile complex.

In further aspects, provided herein are novel nano-sized vesicles comprising of bolaamphiphilic compounds.

In certain aspects, provided herein are novel bolaamphiphile complexes comprising one or more bolaamphiphilic compounds and a biologically active compound.

In one embodiment, the biologically active compound is a compound active against ALS. In another embodiment, the biologically active compound is an analgesic compound.

In further aspects, provided herein are novel formulations of biologically active compounds with one or more bolaamphiphilic compounds or with bolaamhphile vesicles.

In another aspect, provided here are methods of delivering biologically active drugs agents into animal or human brain. In one embodiment, the method comprises the step of administering to the animal or human a pharmaceutical composition comprising of a bolaamphiphile complex; and wherein the bolaamphiphile complex comprises one or more bolaamphiphilic compounds and a compound active against ALS. In one particular embodiment, the biologically active compound is an analgesic compound.

In one embodiment, the bolaamphiphilic complex comprises one bolaamphiphilic compound. In another embodiment, the bolaamphiphilic complex comprises two bolaamphiphilic compounds.

In one embodiment, the bolaamphiphilic compound consists of two hydrophilic headgroups linked through a long hydrophobic chain. In another embodiment, the hydrophilic headgroup is an amino containing group. In a specific embodiment, the hydrophilic headgroup is a tertiary or quaternary amino containing group.

In one particular embodiment, the bolaamphiphilic compound is a compound according to formula I:

HG²L¹-HG¹ I

or a pharmaceutically acceptable salt, solvate, hydrate, prodrug, stereoisomer, tautomer, isotopic variant, or N-oxide thereof, or a combination thereof;
wherein:

each HG¹ and HG² is independently a hydrophilic head group; and

L¹ is alkylene, alkenyl, heteroalkylene, or heteroalkenyl linker; unsubstituted or substituted with C₁-C₂₀ alkyl, hydroxyl, or oxo.

In one embodiment, the pharmaceutically acceptable salt is a quaternary ammonium salt.

In one embodiment, with respect to the bolaamphiphilic compound of formula I, L¹ is heteroalkylene, or heteroalkenyl linker comprising C, N, and O atoms; unsubstituted or substituted with C₁-C₂₀ alkyl, hydroxyl, or oxo.

In another embodiment, with respect to the bolaamphiphilic compound of formula I, L¹ is

—O-L²-C(O)—O—(CH₂)ₙ₄—O—C(O)-L³-O—, or

—O-L²-C(O)—O—(CH₂)ₙ₅—O—C(O)—(CH₂)ₙ₆—,
and wherein each L² and L³ is C₄-C₂₀ alkenyl linker; unsubstituted or substituted with C₁-C₈ alkyl or hydroxy;
and n4, n5, and n6 is independently an integer from 4-20.

In one embodiment, each L² and L³ is independently —C(R¹)—C(OH)—CH₂—(CH═CH)—(CH₂)ₙ₇—; R¹ is C₁-C₈ alkyl, and n7 is independently an integer from 4-20.

In another embodiment, with respect to the bolaamphiphilic compound of formula I, L¹ is O—(CH₂)ₙ₁—O—C(O)—(CH₂)ₙ₂—C(O)—O—(CH₂)ₙ₃—O—.

In another embodiment, with respect to the bolaamphiphilic compound of formula I, L¹ is

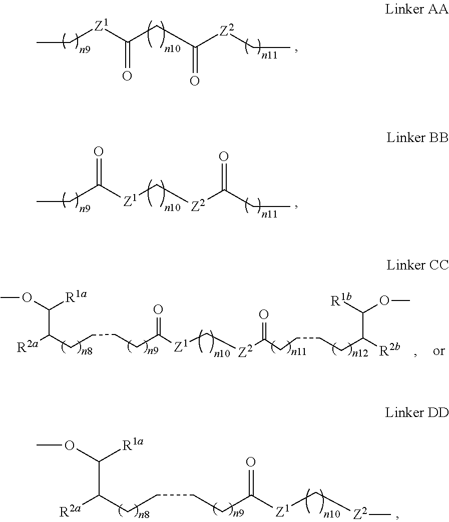

wherein:
each Z¹ and Z² is independently —C(R³)₂—, —N(R³)— or —O—;
each R^{1a}, R^{1b}, R³, and R⁴ is independently H or C₁-C₈ alkyl;
each R^{2a} and R^{2b} is independently H, C₁-C₈ alkyl, OH, or alkoxy;
each n8, n9, n11, and n12 is independently an integer from 1-20;
n10 is an integer from 2-20; and
each dotted bond is independently a single or a double bond.
and wherein each methylene carbon is unsubstituted or substituted with C₁-C₄ alkyl; and each n1, n2, and n3 is independently an integer from 4-20.

In one embodiment, with respect to the bolaamphiphilic compound of formula I, the bolaamphiphilic compound is a compound according to formula II, III, IV, V, or VI:

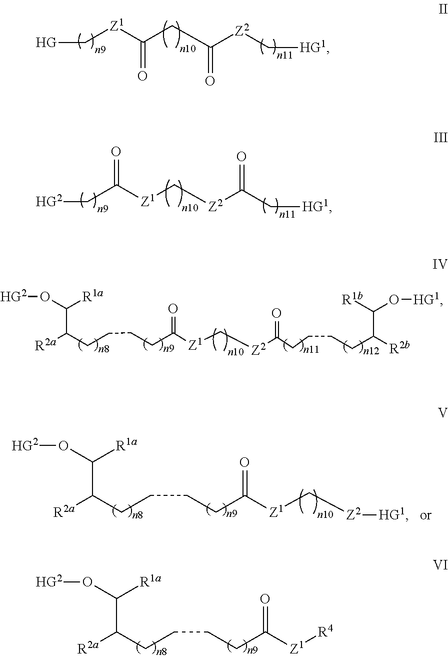

or a pharmaceutically acceptable salt, solvate, hydrate, prodrug, stereoisomer, tautomer, isotopic variant, or N-oxide thereof, or a combination thereof;
wherein:
   each HG¹ and HG² is independently a hydrophilic head group;
   each Z¹ and Z² is independently —C(R³)₂—, —N(R³)— or —O—;
   each R^{1a}, R^{1b}, R³, and R⁴ is independently H or C₁-C₈ alkyl;
   each R^{2a} and R^{2b} is independently H , C₁-C₈ alkyl, OH, alkoxy, or O—HG¹ or O—HG²;
   each n8, n9, n11, and n12 is independently an integer from 1-20;
   n10 is an integer from 2-20; and
   each dotted bond is independently a single or a double bond.

In one embodiment, with respect to the bolaamphiphilic compound of formula II, III, IV, V, or VI, each n9 and n11 is independently an integer from 2-12. In another embodiment, n9 and n11 is independently an integer from 4-8. In a particular embodiment, each n9 and n11 is 7 or 11.

In one embodiment, with respect to the bolaamphiphilic compound of formula II, III, IV, V, or VI, each n8 and n12 is independently 1, 2, 3, or 4. In a particular embodiment, each n8 and n12 is 1.

In one embodiment, with respect to the bolaamphiphilic compound of formula II, III, IV, V, or VI, each R^{2a} and R^{2b} is independently H, OH, or alkoxy. In another embodiment, each R^{2a} and R^{2b} is independently H, OH, or OMe. In another embodiment, each R^{2a} and R^{2b} is independently-O—HG¹ or O—HG². In a particular embodiment, each R^{2a} and R^{2b} is OH.

In one embodiment, with respect to the bolaamphiphilic compound of formula II, III, IV, V, or VI, each R^{1a} and R^{1b} is independently H, Me, Et, n-Pr, i-Pr, n-Bu, i-Bu, sec-Bu, n-pentyl, isopentyl, n-hexyl, n-heptyl, or n-octyl. In a particular embodiment, each R^{1a} and R^{1b} is independently n-pentyl.

In one embodiment, with respect to the bolaamphiphilic compound of formula II, III, IV, V, or VI, each dotted bond is a single bond. In another embodiment, each dotted bond is a double bond.

In one embodiment, with respect to the bolaamphiphilic compound of formula II, III, IV, V, or VI, n10 is an integer from 2-16. In another embodiment, n10 is an integer from 2-12. In a particular embodiment, n10 is 2, 4, 6, 8, 10, 12, or 16.

In one embodiment, with respect to the bolaamphiphilic compound of formula IV, R⁴ is H, Me, Et, n-Pr, i-Pr, n-Bu, i-Bu, sec-Bu, n-pentyl, or isopentyl. In another embodiment, R⁴ is Me, or Et. In a particular embodiment, R⁴ is Me.

In one embodiment, with respect to the bolaamphiphilic compound of formula II, III, IV, V, or VI, each Z¹ and Z² is independently C(R³)₂—, or —N(R³)—. In another embodiment, each Z¹ and Z² is independently C(R³)₂—, or —N(R³)—; and each R³ is independently H, Me, Et, n-Pr, i-Pr, n-Bu, i-Bu, sec-Bu, n-pentyl, or isopentyl. In a particular embodiment, R³ is H.

In one embodiment, with respect to the bolaamphiphilic compound of formula II, III, IV, V, or VI, each Z¹ and Z² is —O—.

In one embodiment, with respect to the bolaamphiphilic compound of formula I, II, III, or IV, each HG¹ and HG² is independently selected from:

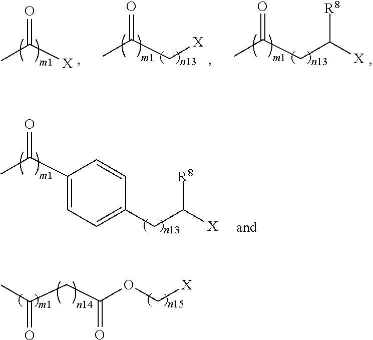

wherein:
X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl; each R^{5c} is independently substituted or unsubstituted C₁-C₂₀ alkyl; each R⁸ is independently H, substituted or unsubstituted C₁-C₂₀ alkyl, alkoxy, or carboxy; m1 is 0 or 1; and
each n13, n14, and n15 is independently an integer from 1-20.

In one embodiment, with respect to the bolaamphiphilic compound of formula I, II, III, or IV, HG¹ and HG² are as defined above, and each m1 is 0.

In one embodiment, with respect to the bolaamphiphilic compound of formula I, II, III, or IV, HG¹ and HG² are as defined above, and each m1 is 1.

In one embodiment, with respect to the bolaamphiphilic compound of formula I, II, III, or IV, HG¹ and HG² are as defined above, and each n13 is 1 or 2.

In one embodiment, with respect to the bolaamphiphilic compound of formula I, II, III, or IV, HG¹ and HG² are as defined above, and each n14 and n15 is independently 1, 2, 3, 4, or 5. In another embodiment, each n14 and n15 is independently 2 or 3.

In one particular embodiment, the bolaamphiphilic compound is a compound according to formula VIIa, VIIb, VIIc, or VIId:

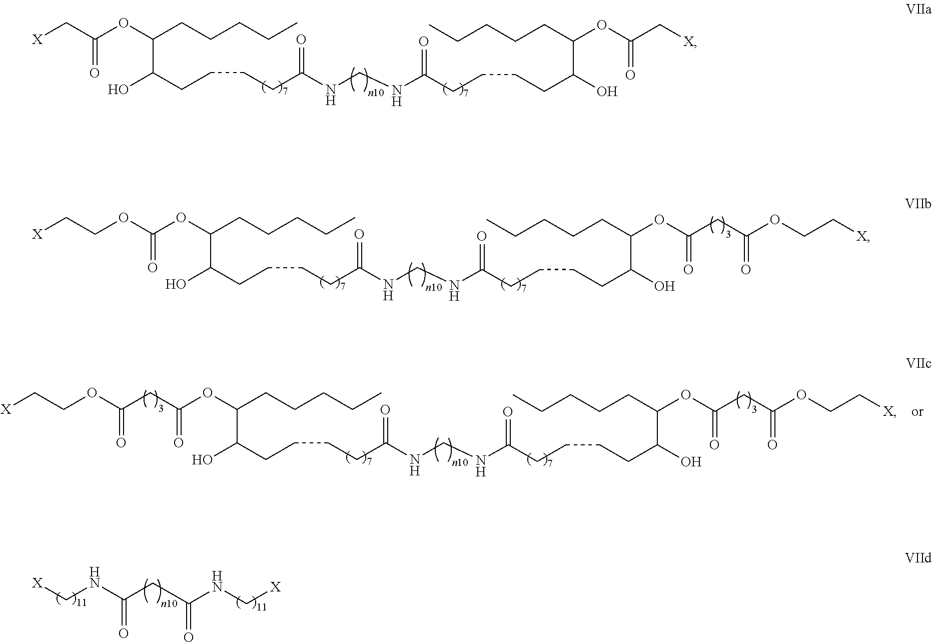

or a pharmaceutically acceptable salt, solvate, hydrate, prodrug, stereoisomer, tautomer, isotopic variant, or N-oxide thereof, or a combination thereof;
wherein:
   each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
      each R^{5c} is independently substituted or unsubstituted C₁-C₂₀ alkyl;
      n10 is an integer from 2-20; and
      each dotted bond is independently a single or a double bond.

In another particular embodiment, the bolaamphiphilic compound is a compound according to formula VIIIa, VIIIb, VIIIc, or VIIId:

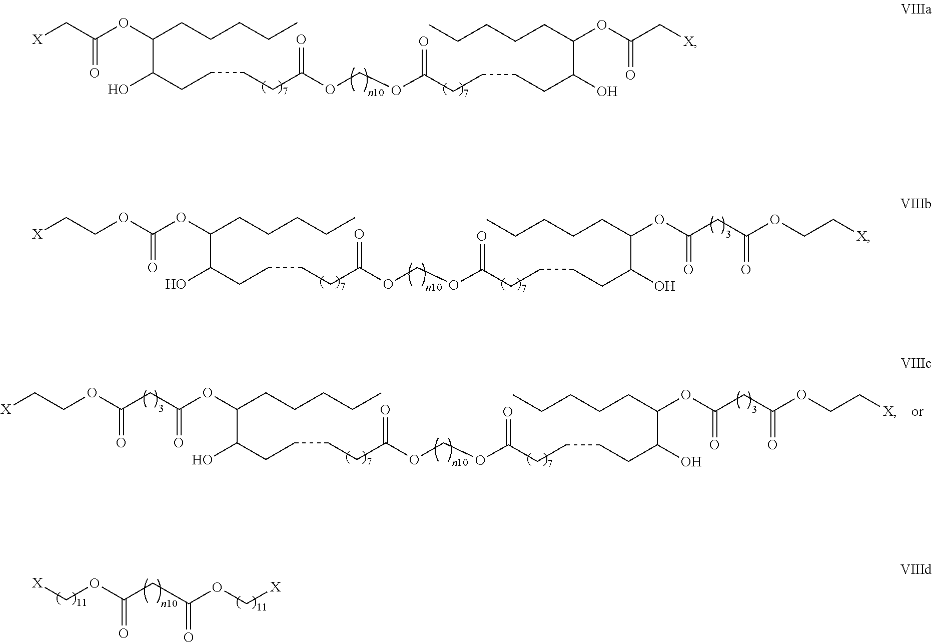

or a pharmaceutically acceptable salt, solvate, hydrate, prodrug, stereoisomer, tautomer, isotopic variant, or N-oxide thereof, or a combination thereof;
wherein:
   each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
      each R^{5c} is independently substituted or unsubstituted C₁-C₂₀ alkyl;
      n10 is an integer from 2-20; and
      each dotted bond is independently a single or a double bond.

In another particular embodiment, the bolaamphiphilic compound is a compound according to formula IXa, IXb, or IXc:

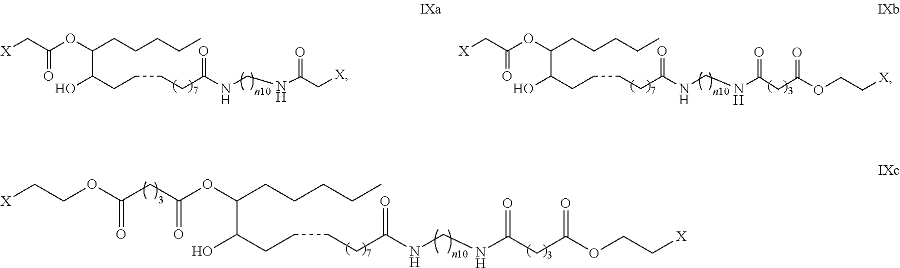

or a pharmaceutically acceptable salt, solvate, hydrate, prodrug, stereoisomer, tautomer, isotopic variant, or N-oxide thereof, or a combination thereof;
wherein:
   each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
      each R^{5c} is independently substituted or unsubstituted C₁-C₂₀ alkyl;
      n10 is an integer from 2-20; and
      each dotted bond is independently a single or a double bond.

In another particular embodiment, the bolaamphiphilic compound is a compound according to formula Xa, Xb, or Xc:

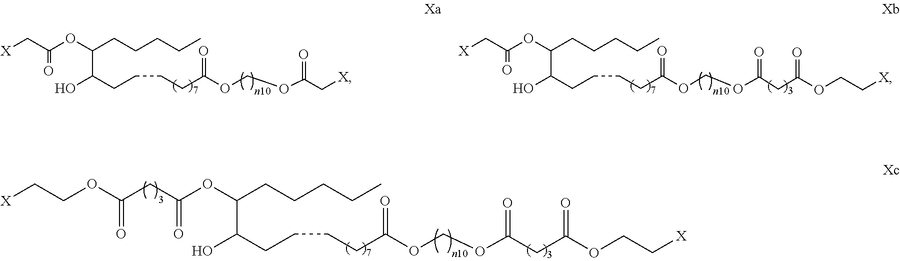

or a pharmaceutically acceptable salt, solvate, hydrate, prodrug, stereoisomer, tautomer, isotopic variant, or N-oxide thereof, or a combination thereof;
wherein:
   each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
   each R^{5c} is independently substituted or unsubstituted C₁-C₂₀ alkyl;
   n10 is an integer from 2-20; and
   each dotted bond is independently a single or a double bond.

In one embodiment, with respect to the bolaamphiphilic compound of formula VIIa-VIId, VIIIa-VIIId, IXa-IXc, or Xa-Xc, each dotted bond is a single bond. In another embodiment, each dotted bond is a double bond.

In one embodiment, with respect to the bolaamphiphilic compound of formula VIIa-VIId, VIIIa-VIIId, IXa-IXc, or Xa-Xc, n10 is an integer from 2-16.

In one embodiment, with respect to the bolaamphiphilic compound of formula VIIa-VIId, VIIIa-VIIId, IXa-IXc, or Xa-Xc, n10 is an integer from 2-12.

In one embodiment, with respect to the bolaamphiphilic compound of formula VIIa-VIId, VIIIa-VIIId, IXa-IXc, or Xa-Xc, n10 is 2, 4, 6, 8, 10, 12, or 16.

In one embodiment, with respect to the bolaamphiphilic compound of formula VIIa-VIId, VIIIa-VIIId, IXa-IXc, or Xa-Xc, each R^{5a}, R^{5b}, and R^{5c} is independently substituted or unsubstituted C₁-C₂₀ alkyl.

In one embodiment, with respect to the bolaamphiphilic compound of formula VIIa-VIId, VIIIa-VIIId, IXa-IXc, or Xa-Xc, each R^{5a}, R^{5b}, and R^{5c} is independently unsubstituted C₁-C₂₀ alkyl.

In one embodiment, with respect to the bolaamphiphilic compound of formula VIIa-VIId, VIIIa-VIIId, IXa-IXc, or Xa-Xc, one of R^{5a}, R^{5b}, and R^{5c} is C₁-C₂₀ alkyl substituted with —OC(O)R⁶; and R⁶ is C₁-C₂₀ alkyl.

In one embodiment, with respect to the bolaamphiphilic compound of formula VIIa-VIId, VIIIa-VIIId, IXa-IXc, or Xa-Xc, two of R^{5a}, R^{5b}, and R^{5c} are independently C₁-C₂₀ alkyl substituted with —OC(O)R⁶; and R⁶ is C₁-C₂₀ alkyl. In one embodiment, R⁶ is Me, Et, n-Pr, i-Pr, n-Bu, i-Bu, sec-Bu, n-pentyl, isopentyl, n-hexyl, n-heptyl, or n-octyl. In a particular embodiment, R⁶ is Me.

In one embodiment, with respect to the bolaamphiphilic compound of formula VIIa-VIId, VIIIa-VIIId, IXa-IXc, or Xa-Xc, one of R^{5a}, R^{5b}, and R^{5c} is C₁-C₂₀ alkyl substituted with amino, alkylamino or dialkylamino.

In one embodiment, with respect to the bolaamphiphilic compound of formula VIIa-VIId, VIIIa-VIIId, IXa-IXc, or Xa-Xc, two of R^{5a}, R^{5b}, and R^{5c} are independently C₁-C₂₀ alkyl substituted with amino, alkylamino or dialkylamino.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted heteroaryl.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted pyridyl.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted monocyclic or bicyclic heterocyclyl.

In one embodiment, with respect to the bolaamphiphilic compound of formula VIIa-VIId, VIIIa-VIIId, IXa-IXc, or Xa-Xc, X is substituted or unsubstituted

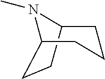

In one embodiment, with respect to the bolaamphiphilic compound of formula VIIa-VIId, VIIIa-VIIId, IXa-IXc, or Xa-Xc, X is

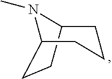

substituted with one or more groups selected from alkoxy, acetyl, and substituted or unsubstituted Ph.

In one embodiment, with respect to the bolaamphiphilic compound of formula VIIa-VIId, VIIIa-VIIId, IXa-IXc, or Xa-Xc, X is

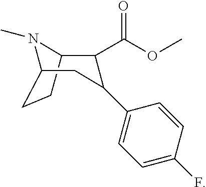 [SMILES: COC(=O)C1C(c2ccc(F)cc2)CC2CCC1N2C]

In one embodiment, with respect to the bolaamphiphilic compound of formula VIIa-VIId, VIIIa-VIIId, IXa-IXc, or Xa-Xc, X is —NMe₂ or —N⁺Me₃.

In one embodiment, with respect to the bolaamphiphilic compound of formula VIIa-VIId, VIIIa-VIIId, IXa-IXc, or Xa-Xc, X is N(Me)-CH₂CH₂—OAc or —N⁺(Me)₂-CH₂CH₂—OAc.

In one embodiment, with respect to the bolaamphiphilic compound of formula VIIa-VIId, VIIIa-VIIId, IXa-IXc, or Xa-Xc, X is a chitosanyl group; and the chitosanyl group is a poly-(D)glucosaminyl group with MW of 3800 to 20,000 Daltons, and is attached to the core via N.

In one embodiment, the chitosanyl group is

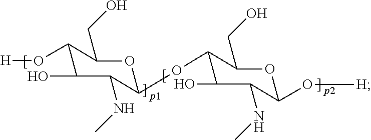

and wherein each pl and p2 is independently an integer from 1-400; and each R^{7a} is H or acyl.

In one embodiment, with respect to the bolaamphiphilic compound of formula VIIa-VIId, VIIIa-VIIId, IXa-IXc, or Xa-Xc, X is a substance P head group. In one embodiment, the substance P head group is bound through the ω-amino group of lysine. In another embodiment, X is —NH—(CH2)₄-C(H)(NH-Pro-Arg)-NH-Pro-Gly-Gly-Phe-Phe-Gly-Leu-Met.

In one embodiment, with respect to the bolaamphiphilic compound of formula VIIa-VIId, VIIIa-VIIId, IXa-IXc, or Xa-Xc, X is a headgroup comprising NK1R antagonist.

In one embodiment, the NK1R antagonist is

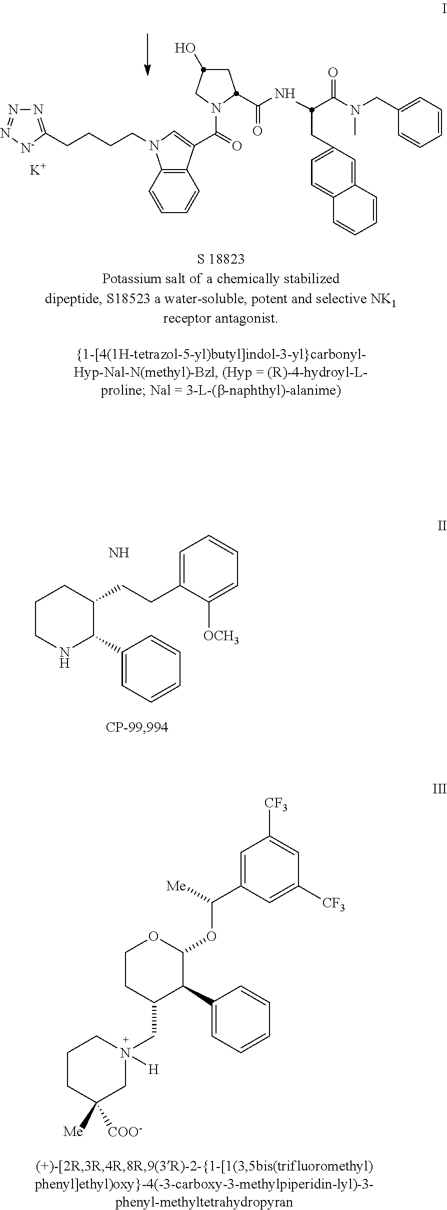

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is a pharmaceutically acceptable salt.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is in a form of a quaternary salt.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is in a form of a quaternary salt with pharmaceutically acceptable alkyl halide or alkyl tosylate.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is any one of the bolaambphilic compounds listed in Table 1.

In another specific aspect, provided herein are methods for incorporating biologically active drugs in the bolavesicles. In one embodiment, the bolavesicle comprises one or more bolaamphilic compounds described herein.

In another specific aspect, provided herein are methods for brain-targeted drug delivery using the bolavesicles incorporated with biologically active drug.

In one particular embodiment, the biologically active drug is kyotorphine or enkephaline.

In one particular embodiment, the biologically active drug is irinotecan (CPT-11 or (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate).

In another specific aspect, provided herein are methods for delivering kyotorphine and enkephaline to the brain.

In another specific aspect, provided herein are methods for delivering CPT-11 to the brain.

In another specific aspect, provided herein are nano-particles, comprising one or more bolaamphiphilic compounds and kyotorphine or enkephaline. In one embodiment, the bolaamphiphilic compounds and kyotorphine or enkephaline are encapsulated within the nano-particle.

In another specific aspect, provided herein are nano-particles, comprising one or more bolaamphiphilic compounds and CPT-11.

In another specific aspect, provided herein are pharmaceutical compositions, comprising a nano-sized particle comprising one or more bolaamphiphilic compounds and kyotorphine, enkephaline, or CPT-11; and a pharmaceutically acceptable carrier.

In another specific aspect, provided herein are methods for treatment or diagnosis of diseases or disorders selected from ALS and related diseases using the nano-particles, pharmaceutical compositions or formulations of the present invention.

In another specific aspect, provided herein are methods for treatment of pain using the nano-particles, pharmaceutical compositions or formulations of the present invention.

The Derivatives and Precursors disclosed can be prepared as illustrated in the Schemes provided herein. The syntheses can involve initial construction of, for example, vernonia oil or direct functionalization of natural derivatives by organic synthesis manipulations such as, but not limiting to, epoxide ring opening. In those processes involving oxiranyl ring opening, the epoxy group is opened by the addition of reagents such as carboxylic acids or organic or inorganic nucleophiles. Such ring opening results in a mixture of two products in which the new group is introduced at either of the two carbon atoms of the epoxide moiety. This provides beta substituted alcohols in which the substitution position most remote from the CO group of the main aliphatic chain of the vernonia oil derivative is arbitrarily assigned as position 1, while the neighboring substituted carbon position is designated position 2. For simplicity purposes only, the Derivatives and Precursors shown herein may indicate structures with the hydroxy group always at position 2 but the Derivatives and Precursors wherein the hydroxy is at position 1 are also encompassed by the invention. Thus, a radical of the formula —CH(OH)—CH(R)— refers to the substitution of —OH at either the carbon closer to the CO group, designated position 2 or to the carbon at position 1. Moreover, with respect to the preparation of symmetrical bolaamphiphiles made via introducing the head groups through an epoxy moiety (e.g., as in vernolic acid) or a double bond (—C═C—) as in mono unsaturated fatty acids (e.g., oleic acid) a mixture of three different derivatives will be produced. In certain embodiments, vesicles are prepared using the mixture of unfractionated positional isomers. In one aspect of this embodiment, where one or more bolas are prepared from vernolic acid, and in which a hydroxy group as well as the head group introduced through an epoxy or a fatty acid with the head group introduced through a double bond (—C═C—), the bola used in vesicle preparation can actually be a mixture of three different positional isomers.

In other embodiments, the three different derivatives are isolated. Accordingly, the vesicles disclosed herein can be made from a mixture of the three isomers of each derivative or, in other embodiments, the individual isomers can be isolated and used for preparation of vesicles.

Symmetrical bolaamphiphiles can form relatively stable self aggregate vesicle structures by the use of additives such as cholesterol and cholesterol derivatives (e.g., cholesterol hemisuccinate, cholesterol oleyl ether, anionic and cationic derivatives of cholesterol and the like), or other additives including single headed amphiphiles with one, two or multiple aliphatic chains such as phospholipids, zwitterionic, acidic, or cationic lipids. Examples of zwitterionic lipids are phosphatidylcholines, phosphatidylethanol amines and sphingomyelins. Examples of acidic amphiphilic lipids are phosphatidylglycerols, phosphatidylserines, phosphatidylinositols, and phosphatidic acids. Examples of cationic amphipathic lipids are diacyl trimethylammonium propanes, diacyl dimethylammonium propanes, and stearylamines cationic amphiphiles such as spermine cholesterol carbamates, and the like, in optimum concentrations which fill in the larger spaces on the outer surfaces, andor add additional hydrophilicity to the particles. Such additives may be added to the reaction mixture during formation of nanoparticles to enhance stability of the nanoparticles by filling in the void volumes of in the upper surface of the vesicle membrane.

Stability of nano vesicles according to the present disclosure can be demonstrated by dynamic light scattering (DLS) and transmission electron microscopy (TEM). For example, suspensions of the vesicles can be left to stand for 1, 5, 10, and 30 days to assess the stability of the nanoparticle solutionsuspension and then analyzed by DLS and TEM.

The vesicles disclosed herein may encapsulate within their core the active agent, which in particular embodiments is selected from peptides, proteins, nucleotides and or non-polymeric agents. In certain embodiments, the active agent is also associated via one or more non-covalent interactions to the vesicular membrane on the outer surface andor the inner surface, optionally as pendant decorating the outer or inner surface, and may further be incorporated into the membrane surrounding the core. In certain aspects, biologically active peptides, proteins, nucleotides or non-polymeric agents that have a net electric charge, may associate ionically with oppositely charged headgroups on the vesicle surface andor form salt complexes therewith.

In particular aspects of these embodiments, additives which may be bolaamphiphiles or single headed amphiphiles, comprise one or more branching alkyl chains bearing polar or ionic pendants, wherein the aliphatic portions act as anchors into the vesicle's membrane and the pendants (e.g., chitosan derivatives or polyamines or certain peptides) decorate the surface of the vesicle to enhance penetration through various biological barriers such as the intestinal tract and the BBB, and in some instances are also selectively hydrolyzed at a given site or within a given organ. The concentration of these additives is readily adjusted according to experimental determination.

In certain embodiments, the oral formulations of the present disclosure comprise agents that enhance penetration through the membranes of the GI tract and enable passage of intact nanoparticles containing the drug. These agents may be any of the additives mentioned above and, in particular aspects of these embodiment, include chitosan and derivatives thereof, serving as vehicle surface ligands, as decorations or pendants on the vesicles, or the agents may be excipients added to the formulation.

In other embodiments, the nanoparticles and vesicles disclosed herein may comprise the fluorescent marker carboxyfluorescein (CF) encapsulated therein while in particular aspects, the nanoparticle and vesicles of the present disclosure may be decorated with one or more of PEG, e.g. PEG2000-vernonia derivatives as pendants. For example, two kinds of PEG-vernonia derivatives can be used: PEG-ether derivatives, wherein PEG is bound via an ether bond to the oxygen of the opened epoxy ring of, e.g., vernolic acid and PEG-ester derivatives, wherein PEG is bound via an ester bond to the carboxylic group of, e.g., vernolic acid.

In other embodiments, vesicles, made from synthetic amphiphiles, as well as liposomes, made from synthetic or natural phospholipids, substantially (or totally) isolate the therapeutic agent from the environment allowing each vesicle or liposome to deliver many molecules of the therapeutic agent. Moreover, the surface properties of the vesicle or liposome can be modified for biological stability, enhanced penetration through biological barriers and targeting, independent of the physico-chemical properties of the encapsulated drug.

In still other embodiments, the headgroup is selected from: (i) choline or thiocholine, O-alkyl, N-alkyl or ester derivatives thereof; (ii) non-aromatic amino acids with functional side chains such as glutamic acid, aspartic acid, lysine or cysteine, or an aromatic amino acid such as tyrosine, tryptophan, phenylalanine and derivatives thereof such as levodopa(3,4-dihydroxy-phenylalanine) and p-aminophenylalanine; (iii) a peptide or a peptide derivative that is specifically cleaved by an enzyme at a diseased site selected from enkephalin, N-acetyl-ala-ala, a peptide that constitutes a domain recognized by beta and gamma secretases, and a peptide that is recognized by stromelysins; (iv) saccharides such as glucose, mannose and ascorbic acid; and (v) other compounds such as nicotine, cytosine, lobeline, polyethylene glycol, a cannabinoid, or folic acid.

In further embodiments, nano-sized particle and vesicles disclosed herein further comprise at least one additive for one or more of targeting purposes, enhancing permeability and increasing the stability the vesicle or particle. Such additives, in particular aspects, may selected from from: (i) a single headed amphiphilic derivative comprising one, two or multiple aliphatic chains, preferably two aliphatic chains linked to a midsectionspacer region such as —NH—(CH₂)₂—N—(CH₂)₂—N—, or —O—(CH₂)₂—N—(CH₂)₂—O—, and a sole headgroup, which may be a selectively cleavable headgroup or one containing a polar or ionic selectively cleavable group or moiety, attached to the N atom in the middle of said midsection. In other asepcts, the additive can be selected from among cholesterol and cholesterol derivatives such as cholesteryl hemmisuccinate; phospholipids, zwitterionic, acidic, or cationic lipids; chitosan and chitosan derivatives, such as vernolic acid-chitosan conjugate, quaternized chitosan, chitosan-polyethylene glycol (PEG) conjugates, chitosan-polypropylene glycol (PPG) conjugates, chitosan N-conjugated with different amino acids, carboxyalkylated chitosan, sulfonyl chitosan, carbohydrate-branched N-(carboxymethylidene)chitosan and N-(carboxymethyl)chitosan; polyamines such as protamine, polylysine or polyarginine; ligands of specific receptors at a target site of a biological environment such as nicotine, cytisine, lobeline, 1-glutamic acid MK801, morphine, enkephalins, benzodiazepines such as diazepam (valium) and librium, dopamine agonists, dopamine antagonists tricyclic antidepressants, muscarinic agonists, muscarinic antagonists, cannabinoids and arachidonyl ethanol amide; polycationic polymers such as polyethylene amine; peptides that enhance transport through the BBB such as OX 26, transferrins, polybrene, histone, cationic dendrimer, synthetic peptides and polymyxin B nonapeptide (PMBN); monosaccharides such as glucose, mannose, ascorbic acid and derivatives thereof; modified proteins or antibodies that undergo absorptive-mediated or receptor-mediated transcytosis through the blood-brain barrier, such as bradykinin B2 agonist RMP-7 or monoclonal antibody to the transferrin receptor; mucoadhesive polymers such as glycerides and steroidal detergents; and Ca²⁺ chelators. The aforementioned head groups on the additives designed for one or more of targeting purposes and enhancing permeability may also be a head group, preferably on an asymmetric bolaamphiphile wherein the other head group is another moiety, or the head group on both sides of a symmetrical bolaamphiphile. In a further embodiment the bolaamphiphile head groups that comprise the vesicles membranes can interact with the active agents to be encapsulated to be delivered in to the brain and brain sites, and or other targeted sites, by ionic interactions to enhance the % encapsulation via complexation and well as passive encapsulation within the vesicles core. Further the formulation may contain other additives within the vehicles membranes to further enhance the degree of encapsulation of the active agents by interactions other than ionic interactions such as polar or hydrophobic interactions.

In other embodiments, nano-sized particle and vesicles discloser herein may comprises at least one biologically active agent is selected from: (i) a natural or synthetic peptide or protein such as analgesics peptides from the enkephalin class, insulin, insulin analogs, oxytocin, calcitonin, tyrotropin releasing hormone, follicle stimulating hormone, luteinizing hormone, vasopressin and vasopressin analogs, catalase, interleukin-II, interferon, colony stimulating factor, tumor necrosis factor (TNF), melanocyte-stimulating hormone, superoxide dismutase, glial cell derived neurotrophic factor (GDNF) or the Gly-Leu-Phe (GLF) families; (ii) nucleosides and polynucleotides selected from DNA or RNA molecules such as small interfering RNA (siRNA) or a DNA plasmid; (iii) antiviral and antibacterial; (iv) antineoplastic and chemotherapy agents such as cyclosporin, doxorubicin, epirubicin, bleomycin, cisplatin, carboplatin, yinca alkaloids, e.g. vincristine, Podophyllotoxin, taxanes, e.g. Taxol and Docetaxel, and topoisomerase inhibitors, e.g. irinotecan, topotecan.

Additional embodiments within the scope provided herein are set forth in non-limiting fashion elsewhere herein and in the examples. It should be understood that these examples are for illustrative purposes only and are not to be construed as limiting in any manner.

### Pharmaceutical Compositions

In another aspect, the invention provides a pharmaceutical composition comprising a pharmaceutically acceptable carrier and a pharmaceutically effective amount of a compound of Formula I or a complex thereof.

When employed as pharmaceuticals, the compounds provided herein are typically administered in the form of a pharmaceutical composition. Such compositions can be prepared in a manner well known in the pharmaceutical art and comprise at least one active compound.

In certain embodiments, with respect to the pharmaceutical composition, the carrier is a parenteral carrier, oral or topical carrier.

The present invention also relates to a compound or pharmaceutical composition of compound according to Formula I; or a pharmaceutically acceptable salt or solvate thereof for use as a pharmaceutical or a medicament.

Generally, the compounds provided herein are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herein can be administered by a variety of routes including oral, rectal, transdermal, subcutaneous, intravenous, intramuscular, and intranasal. Depending on the intended route of delivery, the compounds provided herein are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The above-described components for orally administrable, injectable, or topically administrable compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

The compounds of this invention can also be administered in sustained release forms or from sustained release drug delivery systems. A description of representative sustained release materials can be found in Remington's Pharmaceutical Sciences.

The present invention also relates to the pharmaceutically acceptable formulations of compounds of Formula I. In certain embodiments, the formulation comprises water. In another embodiment, the formulation comprises a cyclodextrin derivative. In certain embodiments, the formulation comprises hexapropyl-β-cyclodextrin. In a more particular embodiment, the formulation comprises hexapropyl-β-cyclodextrin (10-50% in water).

The present invention also relates to the pharmaceutically acceptable acid addition salts of compounds of Formula I. The acids which are used to prepare the pharmaceutically acceptable salts are those which form non-toxic acid addition salts, i.e. salts containing pharmacologically acceptable aniovs such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The following formulation examples illustrate representative pharmaceutical compositions that may be prepared in accordance with this invention. The present invention, however, is not limited to the following pharmaceutical compositions.

### Formulation 1—Injection

A compound of the invention may be dissolved or suspended in a buffered sterile saline injectable aqueous medium to a concentration of approximately 5 mg/mL.

### Methods of Treatment

Bolaamphiphilic vesicles (bolavesicles) may have certain advantages over conventional liposomes as potential vehicles for drug delivery. Bolavesicles have thinner membranes than comparable liposomal bilayer, and therefore possess bigger inner volume and hence higher encapsulation capacity than liposomes of the same diameter. Moreover, bolavesicles are more physically-stable than conventional liposomes, but can be destabilized in a triggered fashion (e.g., by hydrolysis of the headgroups using a specific enzymatic reaction) thus allowing controlled release of the encapsulated material at the site of action (i.e., drug targeting).

Thus, various biologically active drug molecueles can be encapsulated in the bolaamphiphilic vesicles and then delivered to the brain in sufficient concentrations for therapeutic use.

The bola vesicles aggregate into encapsulating monolayer membranes which, together with functional surface groups, provide vesicle stability, penetrability through the BBB and a controlled release mechanism that enables the release of the encapsulated drug primarily in the brain.

The novel nanovesicles can encapsulates drugs, gets through the blood-brain barrier (BBB) and releases the drug in the brain. A major factor limiting the efficacy of some chemotherapeutical agents that are potentially effective in the treatment of malignant gliomas, particularly glioblastoma multiforme (GBM), is that most drugs cannot cross the BBB. A study from Duke University showed that, out of 40 drugs tested, CPT-11 (Irinotecan, used for solid tumors) was the most potent chemotherapeutic agent against patients' gliomas implanted in mice, and was effective against every tumor tested.

However, attempts to treat GBM patients with CPT-11 were unsuccessful because very little gets through the BBB and reaches the tumor. Hence, CPT-11 encapsulated within bola vesicles, can penetrate the brain via the intense capillary network that supplies blood to the brain and can release CPT-11 upon reaching tumor cells. Thus, it would be effective in treating GBM. The efficacy of CPT-11 delivered by bola vesicles may be further increased by administering it with oral temozolamide which, in combination with radiotherapy, prolongs survival by months and, based on literature, acts synergistically with CPT-11 to kill gliomas.

In still further embodiments, the present disclosure also provides nano vesciles prepared from bolaamphiphilic compounds comprising encapsulated cyclodextrin derivatives as well as compositions comprising same and uses thereof.

More specifically, the present disclosure is directed to encapsulation of cyclodextrins within the aqueous core of the bolaamphiphilic vesicles described herein, which are designed to be administered systematically (e.g., intravenous, Intraperitoneal injection (IP) and oral) and delivery the drug or active agents into the CNSbrain and spinal chord.

Three illustrative aspects of these embodiments include: 1) delivery of empty cyclodextrins and cyclodextrins derivatives by bolaamphiphile vesicles to the brain (CNS) after systemic administration for the treatment of Niemann-Pick type C disease; (2) selective delivery to the brain (CNS) or spinal cord hydrophobic lipophilic drugs or active agents with low water solubility by the encapsulation of the said drug or active agent in cyclodextrin or cyclodextrins derivatives which are then encapsulated within bolaamphiphile vesicles with the characteristics needed to deliver the drug or active agents into the CNSbrain or spinal cord via systemic administration. In this way the total amount of drug or active agent per vesicle is increased as the active agent is not only encapsulated within the lipophilic membrane of the vesicle but also within the vesicle core which contains the water soluble cyclodextrins within the hydrophobic cavity of cyclodextrins the hydrophobiclipophilic active agent is encapsulated; and (3) in one aspect, embodiment 2 is used to delivery calcium channel blockers and activators to the CNS and spinal cord. Calcium channel blockers and activators are often low or non water soluble and their delivery to the CNS is problematic as either they cannot penetrate the CNS andor a relatively high concentrations must be systematically administered which causes significant toxic side effects. As described herein, calcium channel blockers and activators are encapsulated in the bolaamphiphile vesicle which can efficiently delivery the active agent or drug into the CNS, via encapsulation in bolaamphiphile membrane and the within cyclodextrins derivatives which are encapsulated within the core of the vesicles and the cyclodextrins is hydrophilic on its external surfaces. Thus the therapeutic dose is reduced and the toxic effective reduced because of targeting to the brain organ by the vesicles which efficiently deliver a high therapeutic dose of the calcium channel blocker or activator to the target site.

The present disclosure describes use of the cyclodextrin derivative hexapropyl-beta-cyclodextrin, and further relates to the pharmaceutically acceptable formulations of compounds of Formula I. In certain embodiments, the cyclodextrin is embedded onto to the surface of the vesicles and it is anchored into the vesicle membrane through the hexylpropyl moiety; i.e., it is not encapsulated within the vesicle core rather attached to the surface, which may block the vesicle's ability to penetrate through biological organs and the amount of agent encapsulated is limited as the amount of cyclodextrin groups on the surface is significantly less than can be encapsulated with the core of the vesicle. Accordingly, the present disclosure further provides approaches for encapsulating the cyclodextrins in the core of the vesicles.

Cyclodextrins are a family of compounds made up of sugar molecules bound together in a ring. The exterior of the ring is hydrophilic and the interior is relatively hydrophobic. In this way the solubility of molecules with that have low water solubility can be improved by their encapsulation within the cyclodextrin ring. They are used in food, pharmaceutical, drug delivery and chemical industries, as well as agriculture and environmental engineering. Cyclodextrins are composed of 5 or more α-D-glucopyranoside units linked 1->4, as in amylose. Typical cyclodextrins contain a number of glucose monomers ranging from six to eight units in a ring, creating a cone shape: (a) Alpha-cyclodextrin: 6-membered sugar ring molecule; (b) β (beta)-cyclodextrin: 7-membered sugar ring molecule, (c) γ (gamma)-cyclodextrin: 8-membered sugar ring molecule, (d) Hydroxypropyl-β-cyclodextrin (HPβCD) and (e) Methyl-β-cyclodextrin. Each of these are encompassed within the present disclosure; also included are other cyclodextrin derivatives known in the art, which may be incorporated with the bolaamphiphilic vesicles of the present invention. In particular for the treatment or prevention of certain diseases that require the removal of cholesterol a preferred embodiment of the present invention is the encapsulation of β-cyclodextrin and methyl-β-cyclodextrin (MβCD). Both β-cyclodextrin and methyl-β-cyclodextrin (MβCD) can remove cholesterol from cultured cells. The methylated form MβCD was found to be more efficient than β-cyclodextrin. The water-soluble MβCD is known to form soluble inclusion complexes with cholesterol, thereby enhancing its solubility in aqueous solution. MβCD is employed for the preparation of cholesterol-free products: the bulky and hydrophobic cholesterol molecule is easily lodged inside cyclodextrin rings that are then removed. MβCD is also employed in research to disrupt lipid rafts by removing cholesterol from membranes. It has also been shown how cyclodextrin assists in moving cholesterol out of lysosomes in Niemann-Pick type C disease and thus treating this disease—Which is a lysosomal storage disease causing progressive deterioration of the nervous system and dementia. It usually affects young children by interfering with their ability to metabolize cholesterol at the cellular level. Numerous research studies have followed showing that hydroxypropyl-β-cyclodextrin (HPβCD) is not simply an agent to solubilize drugs but has powerful pharmacological properties.

It is however difficult to get high concentrations of both β-cyclodextrin and methyl-β-cyclodextrin (MβCD) into the CNS to treat Niemann-Pick type C disease after systemic administration because of limit biological stability in the blood and poor penetration through the blood brain barrier (BBB). Also, the delivery of empty cyclodextrins is of low efficiency as lipids and cholesterol and other lipophilic molecules found in the blood and cell membranes can fill the cyclodextrin core and reducing the number of empty cyclodextrins reaching the disease site. One approach to overcoming these issues would be to cyclodextrins in the design of novel drug delivery with liposomes, which are limited as they are made from single headed phospholipids. Encapsulating the cyclodextrins within the liposomes results in the cyclodextrins extracting phospholipids and the cholesterol and cholesterol derivative additives used to form the liposomal membrane. Thus limiting the liposomes shelf life and biological stability. And in addition much of the efficacy of the cyclodextrins is lost by the filling of its internal hydrophobic core by the phospholipids and cholesterol additives.

The present inventors have surprising found that with the use of bolaamphiphiles of specific molecular design, vesicles with encapsulated empty cyclodextrins can be prepared such that the vesicles which can be used to deliver the cyclodextrins to the CNDs or spinal cord after systemic administration are stable and do not fill the hydrophobic core of the cyclodextrin with the vesicles components. The bolaamphiphiles used in forming the vesicles have two relatively large ionic head groups vesicles to prevent bolas filling interior of the cyclodextrins. Thus the vesicle's bolaamphiphiles molecular structure with two “large” terminal hydrophilic head groups will prevent their uptake within the cyclodextrin ensuring vesicle stability and cyclodextrin efficacy. The cyclodextrins water solubility allows for high therapeutic concentrations in the aqueous core of our vesicles, and its interactions with the interior bolaamphiphile head groups that comprise the vesicles membranes further enhancing loading within the vesicle.

It has also been discovered the inventors' bolaamphiphilic (bola) vesicles can be used to encapsulate cyclodextrins (CDs) with encapsulated hydrophobic or low water soluble drugs. This combinations takes advantage of inventors' bola vesicles to delivery drugs to target sites and organs such as the brains and specific sites within the brain and the high encapsulation efficiency that can be achieved for low water soluble drugs which are encapsulated with this invention both within the bola membrane and within the water core of the vesicle by the water soluble CD contain the active agent within its hydrophobic interior.

In contrast, liposomes entrap hydrophilic drugs in the aqueous phase and hydrophobic drugs in the lipid bilayers and retain drugs en route to their destination. Major problems encountered with these vesicular systems appears during their preparation and results from a low water solubility of the drug is rapidly released in the presence of plasma leading to either a low yield in drug loading, or a slow or incomplete release rate of the drug. These limitations are overcome using the presently-described approach involving entrapping the CD-drug complexes into vesciles, which combines the advantages of both CDs (such as increasing the solubility of drugs) and liposomes (such as targeting of drugs) into a single system and thus circumvents the problems associated with each system. By forming water soluble complexes, CDs would allow insoluble drugs to accommodate in the aqueous phase of vesicles and thus potentially increase drug-to-lipid mass ratio levels, enlarge the range of insoluble drugs amenable for encapsulation (i.e., membrane-destabilizing agents), allow drug targeting, and reduce drug toxicity.

Potential problems associated with intravenous administration of CD complexes (such as their rapid removal into urine and toxicity to kidneys, especially after chronic use), can be circumvented by their entrapment in liposomes. Liposomal entrapment can also alter the pharmacokinetics of inclusion complexes. Liposomal entrapment drastically reduced the urinary loss of HP-b-CDdrug complexes but augmented the uptake of the complexes by liver and spleen, where after liposomal disintegration in tissues, drugs were metabolized at rates dependent on the stability of the complexes.

Liposome's drug delivery systems are however not efficient active targeting drug delivery systems because of their relatively poor intact penetration through biological barriers, lack of stability needed for an active delivery into specific organs and to sites within these organs and the inability to combine stability with an efficient release mechanism at the target site. The bola vesicles of the present disclosure can achieve these objectives using bolas with specific molecular structures that with other components that can self-assemble into multifunctional particles with a high encapsulation efficiency, biological stability and intact penetration through biological barriers, targeting and an efficient disruption mechanism at the target site. In combining these properties with the encapsulation of CD containing a hydrophobic or low water soluble agent or drug we can achieve a very high encapsulation loading and efficient targeting to a given site of the encapsulated drug.

In one embodiment, calcium channel blockers and activators are delivered to the CNS and spinal cord. Calcium (Ca) channel blockers and activators are often non water soluble and their delivery to the CNS is problematic as either they cannot penetrate the CNS andor a relatively high concentrations must be systematically administered which causes significant toxic side effects. The present disclosure describes encapsulation of calcium channel blockers and activators in the bolaamphiphile vesicles which can efficiently delivery the active agent or drug into the CNS, via encapsulation in bolaamphiphile membrane and the within cyclodextrins derivatives which are encapsulated within the core of the vesicles and the cyclodextrins is hydrophilic. Thus the therapeutic dose is reduced and the toxic effective reduced because of targeting to the brain organ by the vesicles which efficiently deliver a high therapeutic dose of the calcium channel blocker or activator to the target site.

The different Ca channel blockers and activators that we can delivery to the CNS are often used for treating non CNS diseases but have beneficial effects on CNs diseases. Examples of such active agents include:

L-type are Ca channel blocker drugs are used as cardiac antiarrhythmics or antihyoertensives, depending on whether the drugs have higher affinity for the heart (the phenylalkylamines, like verapamil), or for the vessels (the dihydropyridines, like nifedipine). Calcium-channels, blockers have an established role in the management of cardiac arrhythmias. They were identified empirically with the idea of achieving selective inhibition of voltage-gated calcium-channels and vasodilatation

Ca Channel control agents for the treatment of an amyloidosis such as Alzheimer's disease comprises administering an inhibitor of the interaction between A.beta. globulomer and the P/Q type voltage-gated presynaptic calcium channel to said subject

Nitrone-based compositions for the prevention and treatment of a variety of ophthalmic diseases or conditions where RPE65 protein isomerohydrolase is implicated.

Nimodipine- is a dihydropyridine Ca channel blocker originally developed for the treatment of high blood pressure.

Calcium channel blockers (calcium antagonists) have been used in an attempt to prevent cerebral vasospasm after injury, maintain blood flow to the brain, and so prevent further damage.

Ca channel active agents tested for the reduction of Parkinson's disease risk that include isradipine, nimodipine, and nifedipine, among others. All are dihydropyridine derivatives, which block so-called L-type calcium channels on smooth muscle, reducing the force of contraction and thus reducing blood pressure. Amlodipine, doesn't readily cross the blood-brain barrier was not evaluated by other but in our encapsulated in our vesicles would readily cross the BBB into the brain and was effective. In particular “the at risk of developing Parkinson's disease should benefit by the use of calcium blockers such as isradipine as it appears that the dopamine-producing cells in the SN begin to disappear well before the onset of symptoms. By the use of our vesicles combined with CD encapsulation we can readily target our vesicles to the regions affect by Parkinson's disease and thus have a highly beneficial effect.

Calcium channel blockers protect neurons by lowering blood pressure and reversing cellular-level calcium channel dysfunction that occurs with age, cerebral infarction, and Alzheimer's disease (AD). The following illustrative Calcium channel blockers can be used in the methods and compositions of the present disclosure: (a) Select dihydropyridines inhibit amyloidogenesis in apolipoprotein E carriers, such as, amlodipine and nilvadipine reduce β-secretase activity and amyloid precursor protein-13 production; nilvadipine and nitrendipine limit β-amyloid protein synthesis in the brain and promote their clearance through the blood-brain barrier; nilvadipine-treated apolipoprotein E carriers experience cognitive stabilization compared with cognitive decreases seen in non-treated subjects; (b) Dihydropyridines can produce therapeutic effects for both AD and cerebrovascular dementia patients, indicating the potential that certain agents in this class have for treating both conditions.

In still further embodiments, the present disclosure also provides embodiments involving forming bola vesicles with a solid particle of a hydrophobic drug comprises dissolving one or more of the bola derivatives and other additives and the water insoluble drug in a water miscible common solvent or solvent combination, and injecting it into an excess of water such that the drug particles precipate out as nano particles within the core of the bola vesicles which self-assemble around the precipitating drug. The “common solvent” refers to a solvent or combination of solvents in which both the amphiphile and the hydrophobic drug dissolve.

In one embodiment, the common solvent is an alkanol such as ethanol or isopropyl alcohol, and the method consists in injecting the alcoholic solution comprising the bola amphiphile and additives and the hydrophobic drug under the surface of an aqueous solvent, whereby the bola amphiphile forms vesicles within the encapsulated space of the bola vesicle the drug precipitates. Typically, this can be achieved by injection of an alcoholic solution through a small bore Hamilton syringe into a well-stirred aqueous solution. In addition to ethanol and isopropyl alcohol, other water-soluble alcohols and water-miscible solvents such as tetrahydrofuran (THF), N-methylpyrrolidone (NMP), dimethylformamide (DMF) and dimethyl sulfoxide (DMSO), or a combination thereof, may be used. The amount of solvent in the aqueous phase should be sufficiently low so as to not disrupt the formed bola vesicles.

The hydrophobic drugs may be from many different categories and in one embodiment these drugs are taken from Ca channel blockers and or activators including those disclosed herein.

An example of the approach is to: Bolaamphiphiles (GLH 19 and GLH 20 in a ratio of 2/1) , cholesterol, and CHEMS (2:1:1 mole ratio) where in the bolas are together at 20 mg and a calcium channel blocker are Amlodipine (20 mg) dissolved in lml ethanolDMSO at a ratio of ½. One ml of nitrogen-purged aqueous media (e.g. water, saline, solute solution, etc) was placed in a 5 ml vial and stirred rapidly using a magnetic stirrer. A fine gauge needle was fitted to a 1 ml glass syringe and used to draw up to 100 .mu.1 of the bola drug solution. The tip of the needle was positioned below the surface of the stirred aqueous solution, and the bola d solution was injected as rapidly as possible into the aqueous media which was kept at room temperature. The bola vesicles were formed immediately with encapsulated solid particles of drug.

The present disclosure further provides (a) surface-targeting mechanisms comprising the use of a tumor specific ligand to target vesicles to brain tumor, (b) membrane release mechanisms involving the design head groups hydrolyzable by Acetyl Cholinesterase (AChE, which is found at high levels outside of GBM cells), (c) core-drug encapsulation, involving loading vesicles with chemotherapeutic that have proven potency against human GBM, but no BBB permeability, (d) administration mechanisms including intravenous and oral routes; and combination therapies.

In other embodiments, the present disclosure provides nano-sized particles comprising multi-headed amphiphiles for targeted drug discovery. In one aspect of such embodiments, that present disclosure provides treatment of brain tumors by IV and oral administration, surface ligands on the vesicle surface for targeting to sites in the brain, release mechanism form the vesicles with acetyl choline groups by acetyl choline esterase, use of surface ligands such as chitosan for enhancing penetration through the BBB, and GI tract. Vesicles useful in these embodiments may comprise, e.g., cholesterol and cholesterol hemisuccinate, and chitosan alkyl conjugates to place chitosan surface groups on the vesicles' surface; such vesicles may comprise bolas with chitosan head groups andor bola conjugates.

In specific aspects of these embodiments, the present disclosure also provides targeting ligands, including the four illustrative ligands described below.

In one aspect, these embodiments include the synthesis of bolas with NK1R-ligand head groups, i.e., GBM tumor cells highly express the neurokinin-1-receptor (NK1R). Accordingly, such tumors are targeted by attaching NK1R ligands to the bola skeletons as head groups. The head groups may be substance P, an endogenous peptide that serves as the natural ligand for NK1R, andor antagonists with high affinity to NK1R. These bolas are used as one of the building blocks in vesicle formulation to decorate the outer surface of the vesicle with a targeting ligand. A substance P- radiolabelled-analog has shown excellent targeting of GBM tumors in patients.

Synthesis of bolas with substance P as the head group is achieved by covalent binding of substance P to fatty acids using standard protein conjugation methodologies, e.g., activation of the carboxylate by N-hydroxy succinimide in the presence of dicyclohexylcarboiimide and subsequent formation of the amidic linkage. The aliphatic-amide products, which are formed, are very stable. For example, selective derivatization of substance P peptide's lysine ω-amino group (as depicted in the scheme below) may be used, since lysine amines are reasonably good nucleophiles above pH 8.0 (pKa=9.18) and react easily with a variety of reagents to form stable bonds, while other amino groups in the peptide are less reactive under these conditions. Fatty acid-substance P conjugates with variations in chain length and saturation of the fatty acid moiety are also synthesized and examined to determine their toxicity and ability to be incorporated into the vesicles.

The following provides an illustrative approach for synthesis of an amphiphilic compound with substance P head group bound through the ω-amino group of lysine:

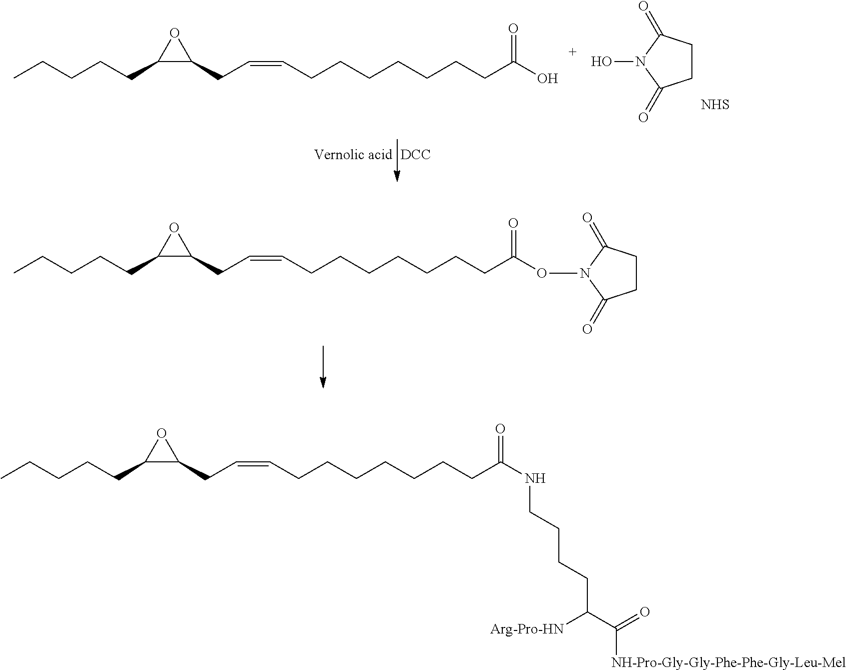

In a second approach, bolas with NK-1-receptor antagonist head groups as the targeting ligand can be synthesized as well as bolas with NK1R antagonists as the targeting ligand can be synthesized. The NK1R antagonists, Peptide I and non-peptide compounds II and III (see below), are used. For the synthesis of the bolas with Peptide I head group, the conjugation is carried out through the nitrogen of the indole ring or through the hydroxyproline residue; for compound II, through the amino group; and for compound III, the fatty acid residue will be attached through the carboxylic group, or alternatively through the amino group. In each case, the site of attachment is chosen based on the results of the targeting efficacy in vitro studies.

The following provides illustrative examples of compounds useful as NK1R antagonists.

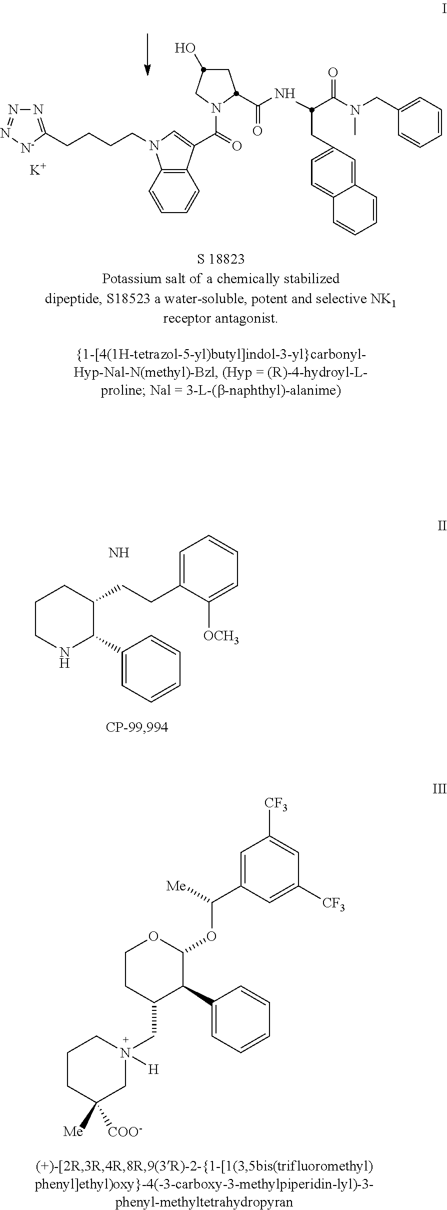

### General Synthetic Procedures

The compounds provided herein can be purchased or prepared from readily available starting materials using the following general methods and procedures. See, e.g., Synthetic Schemes below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography or HPLC. The following schemes are presented with details as to the preparation of representative substituted biarylamides that have been listed herein. The compounds provided herein may be prepared from known or commercially available starting materials and reagents by one skilled in the art of organic synthesis.

The enantiomerically pure compounds provided herein may be prepared according to any techniques known to those of skill in the art. For instance, they may be prepared by chiral or asymmetric synthesis from a suitable optically pure precursor or obtained from a racemate by any conventional technique, for example, by chromatographic resolution using a chiral column, TLC or by the preparation of diastereoisomers, separation thereof and regeneration of the desired enantiomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, NY, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, N.Y.).

In certain embodiments, an enantiomerically pure compound of formula (1) may be obtained by reaction of the racemate with a suitable optically active acid or base. Suitable acids or bases include those described in Bighley et al., 1995, Salt Forms of Drugs and Adsorption, in Encyclopedia of Pharmaceutical Technology, vol. 13, Swarbrick & Boylan, eds., Marcel Dekker, New York; ten Hoeve & H. Wynberg, 1985, Journal of Organic Chemistry 50:4508-4514; Dale & Mosher, 1973, J. Am. Chem. Soc. 95:512; and CRC Handbook of Optical Resolution via Diastereomeric Salt Formation, the contents of which are hereby incorporated by reference in their entireties.

Enantiomerically pure compounds can also be recovered either from the crystallized diastereomer or from the mother liquor, depending on the solubility properties of the particular acid resolving agent employed and the particular acid enantiomer used. The identity and optical purity of the particular compound so recovered can be determined by polarimetry or other analytical methods known in the art. The diasteroisomers can then be separated, for example, by chromatography or fractional crystallization, and the desired enantiomer regenerated by treatment with an appropriate base or acid. The other enantiomer may be obtained from the racemate in a similar manner or worked up from the liquors of the first separation.

In certain embodiments, enantiomerically pure compound can be separated from racemic compound by chiral chromatography. Various chiral columns and eluents for use in the separation of the enantiomers are available and suitable conditions for the separation can be empirically determined by methods known to one of skill in the art. Exemplary chiral columns available for use in the separation of the enantiomers provided herein include, but are not limited to CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

Abbreviations
BBB, blood brain barrier
BCECs, brain capillary endothelial cells
CF, carboxyfluorescein
CHEMS, cholesteryl hemisuccinate
CHOL, cholesterol
Cryo-TEM, Cryo-transmission electron microscope
DAPI, 4′,6-diamidino-2-phenylindole
DDS, drug delivery system
DLS, dynamic light scattering
DMPC, 1,2-dimyristoyl-sn-glycero-3-phosphocholine
DMPE, 1,2-dimyristoyl-sn-glycero-3-phosphoethanolamine
DMPG, 1,2-dimyristoyl-sn-glycero-3-phospho-(1′-rac-glycerol)
EPR, electron paramagnetic resonance
FACS, fluorescence-activated cell sorting
FCR, fluorescence colorimetric response
GUVs, giant unilamellar vesicles
HPLC, high performance liquid chromatography
IR, infrared
MNPs, Magnetic Nanoparticles
MRI, magnetic resonance imaging
NMR, nuclear magnetic resonance
NPs, nanoparticles
PBS, phosphate buffered saline
PC, phosphatidylcholine
PDA, polydiacetylene.
TMA-DPH, 1-(4 trimethylammoniumphenyl)-6-phenyl-1,3,5-hexatriene

### EXAMPLE 1

### Bolaamphiphile Synthesis

The boloamphiphles or bolaamphiphilic compounds of the invention can be synthesized following the procedures described previously (see below).

Briefly, the carboxylic group of methyl vernolate or vernolic acid was interacted with aliphatic diols to obtain bisvernolesters. Then the epoxy group of the vernolate moiety, located on C12 and C13 of the aliphatic chain of vernolic acid, was used to introduce two ACh headgroups on the two vicinal carbons obtained after the opening of the oxirane ring. For GLH-20 (Table 1), the ACh head group was attached to the vernolate skeleton through the nitrogen atom of the choline moiety. The bolaamphiphile was prepared in a two-stage synthesis: First, opening of the epoxy ring with a haloacetic acid and, second, quaternization with the N,N-dimethylamino ethyl acetate. For GLH-19 (Table 1) that contains an ACh head group attached to the vernolate skeleton through the acetyl group, the bolaamphiphile was prepared in a three-stage synthesis, including opening of the epoxy ring with glutaric acid, then esterification of the free carboxylic group with N,N-dimethyl amino ethanol and the final product was obtained by quaternization of the head group, using methyl iodide followed by exchange of the iodide ion by chloride using an ion exchange resin.

Each bolaamphiphile was characterized by mass spectrometry, NMR and IR spectroscopy. The purity of the two bolaamphiphiles was >97% as determined by HPLC.

Materials. Iron(III) acetylacetonate (Fe(acac)₃), diphenyl ether, 1,2-hexadecanediol, oleic acid, oleylamine, and carboxyfluorescein (CF) were purchased from Sigma Aldrich (Rehovot, Israel). Chloroform and ethanol were purchased from Bio-Lab Ltd. Jerusalem, Israel. 1,2-dimyristoyl-sn-glycero-3-phospho-(1′-rac-glycerol) (DMPG), 1,2-dimyristoyl-sn-glycero-3-phosphoethanolamine (DMPE), 1,2-dimyristoyl-sn-glycero-3-phosphocholine (DMPC), cholesterol (CHOL), cholesteryl hemisuccinate (CHEMS) were purchased from Avanti Lipids (Alabaster, Ala., USA), The diacetylenic monomer 10,12-tricosadiynoic acid was purchased from Alfa Aesar (Karlsruhe, Germany), and purified by dissolving the powder in chloroform, filtering the resulting solution through a 0.45 nm nylon filter (Whatman Inc., Clifton, N.J., USA), and evaporation of the solvent. 1-(4 trimethylammoniumphenyl)-6-phenyl-1,3,5-hexatriene (TMA-DPH) was purchased from Molecular Probes Inc. (Eugene, Oreg., USA).

### Synthesis of Representative Bolaamphiphilic Compounds

The synthesis bolaamphiphilic compounds of this invention can be carried out in accordance with the methods described previously (Chemistry and Physics of Lipids 2008, 153, 85-97; Journal of Liposome Research 2010, 20, 147-59; WO2002055011; WO2003047499; or WO2010128504) and using the appropriate reagents, starting materials, and purification methods known to those skilled in the art. Table 1 lists the representative bolaamphiphilic compounds of the invention.

TABLE 1
Representative Bolaamphiphiles

| # | Structure |
|---|---|
| GLH- 3 | 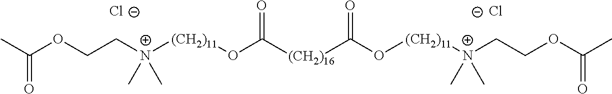 |
| GLH- 4 | 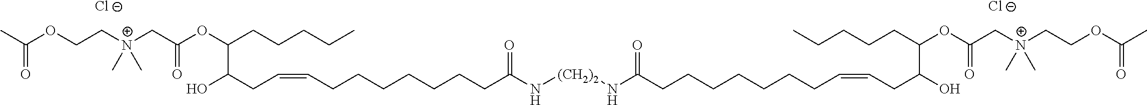 |
| GLH- 5 | 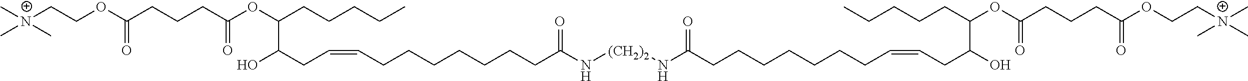 |
| GLH- 6^{a} | 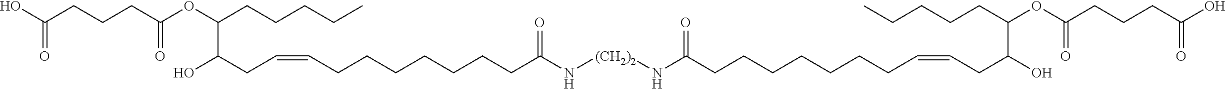 |
| GLH- 7 | 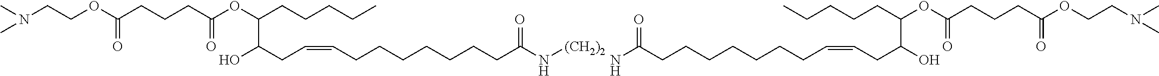 |
| GLH- 8 | 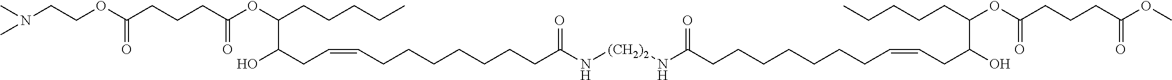 |
| GLH- 9 | 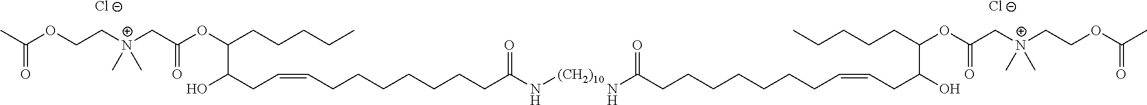 |
| GLH- 10 | 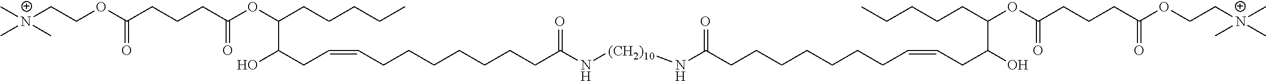 |
| GLH- 11 | 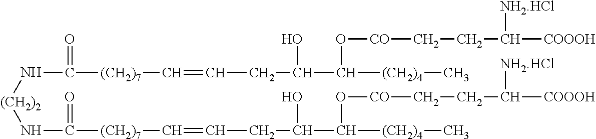 |
| GLH- 12^{a} | 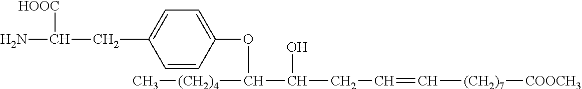 |
| GLH- 13^{a} | 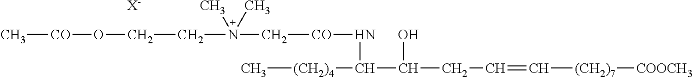 |
| GLH- 13^{a} | 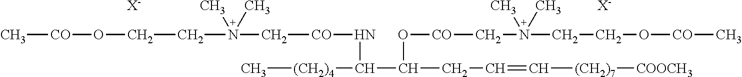 |
| GLH- 14 | 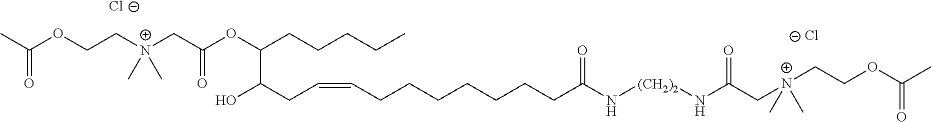 |
| GLH- 15 | 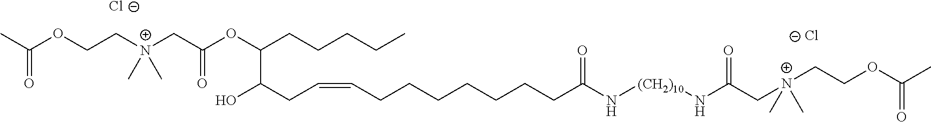 |
| GLH- 16 | 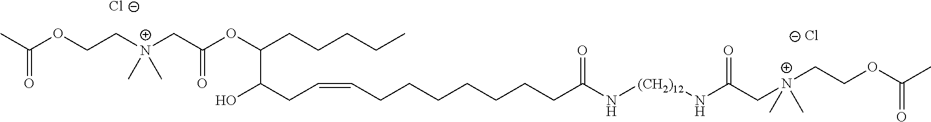 |
| GLH- 17 | 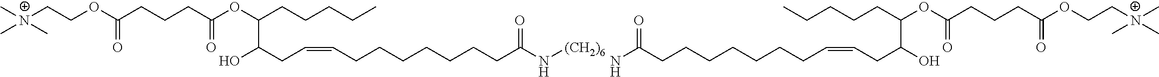 |
| GLH- 18 | 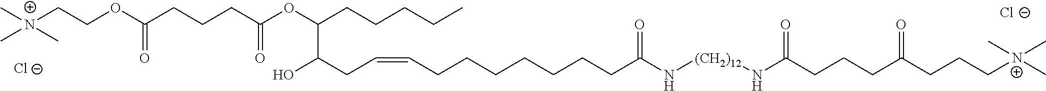 |
| GLH- 19 | 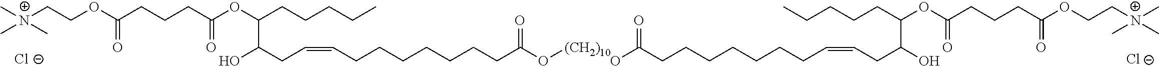 |
| GLH- 20 | 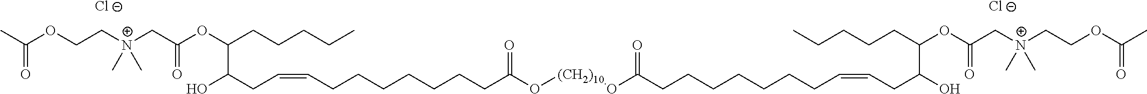 |
| GLH- 21 | 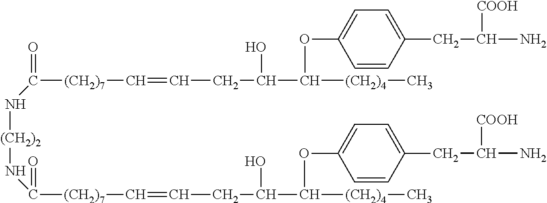 |
| GLH- 22 | 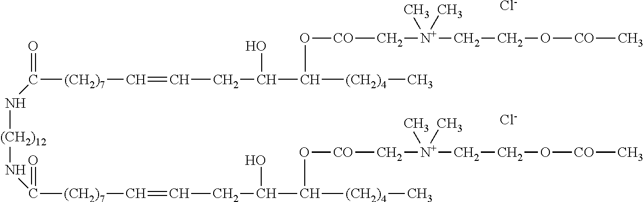 |
| GLH- 23 | 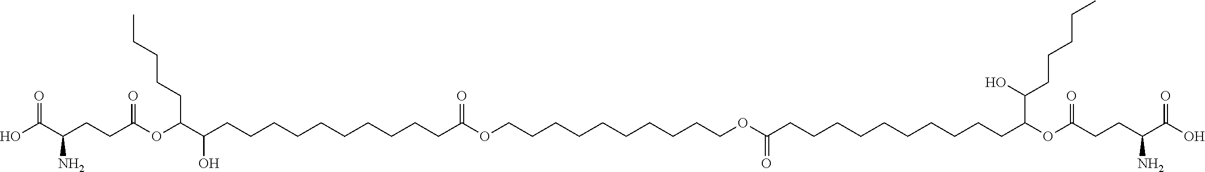 [SMILES: CCCCCC(O)C(CCCCCCCCCCC(=O)OCCCCCCCCCCOC(=O)CCCCCCCCCCC(O)C(CCCCC)OC(=O)CC[C@@H](N)C(=O)O)OC(=O)CC[C@H](N)C(=O)O] |
| GLH- 24 | 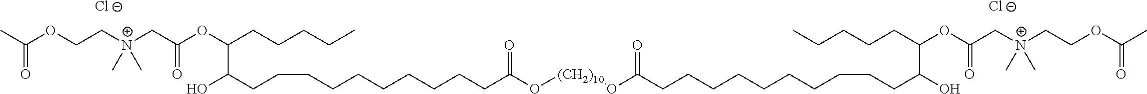 |
| GLH- 25 | 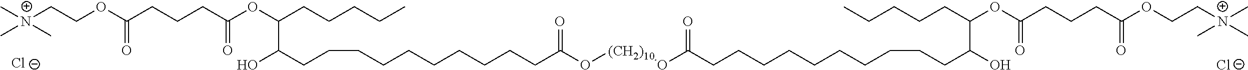 |
| GLH- 26 | 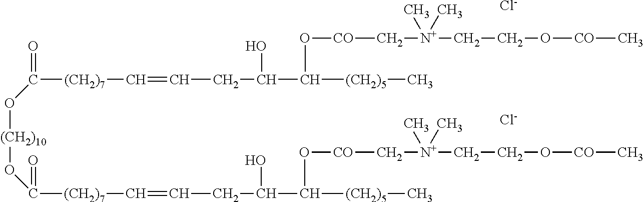 |
| GLH- 27 | 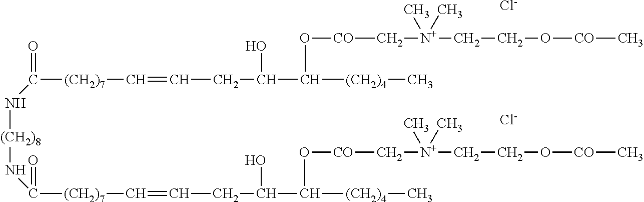 |
| GLH- 28 | 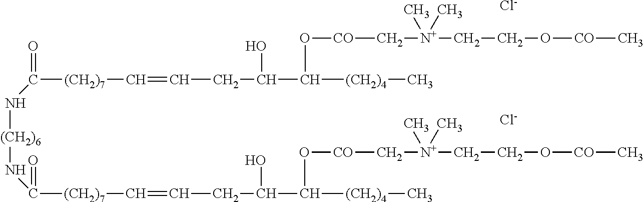 |
| GLH- 29 | 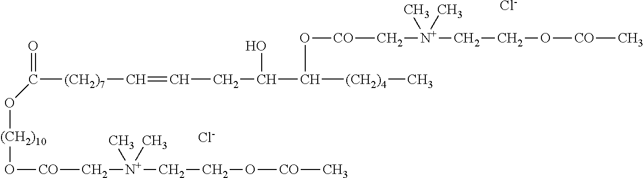 |
| GLH- 30 | 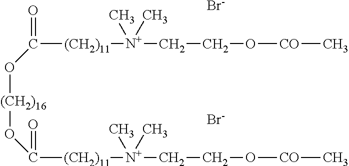 |
| GLH- 30 | 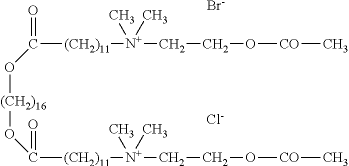 |
| GLH- 31 | 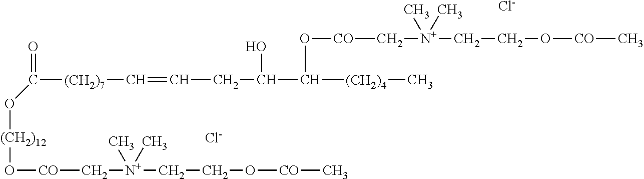 |
| GLH- 32 | 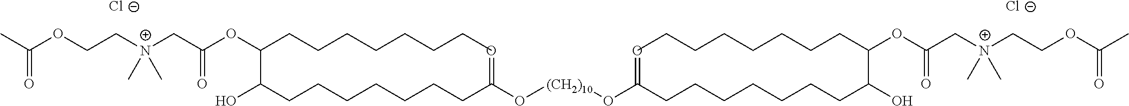 |
| GLH- 33 | 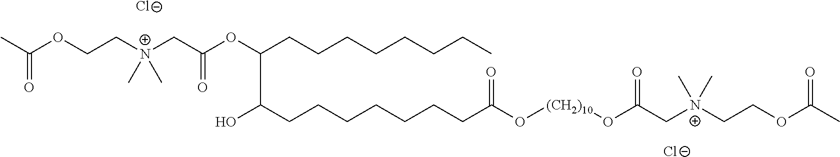 |
| GLH- 34 | 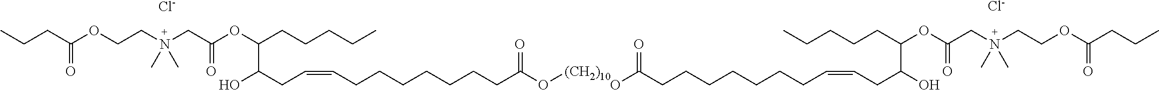 |
| GLH- 35 | 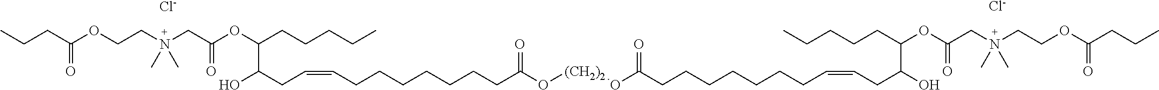 |
| GLH- 36 | 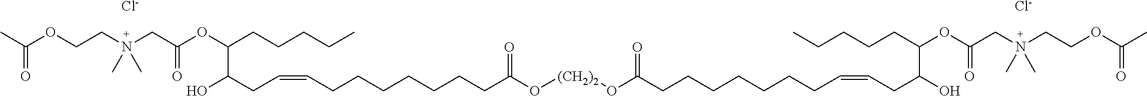 |
| GLH- 37 | 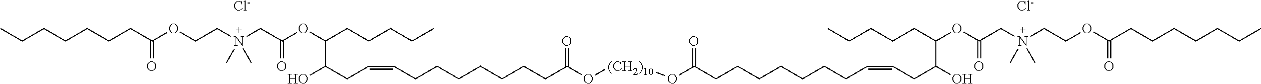 |
| GLH- 38 | 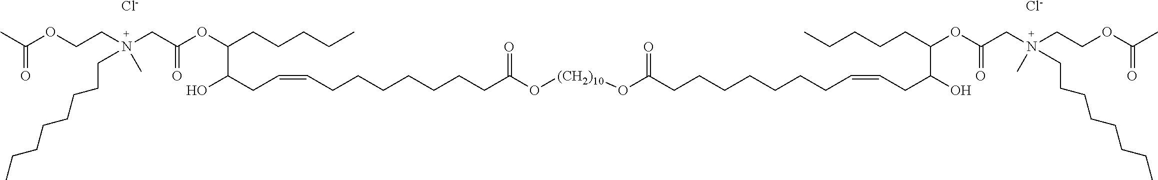 |
| GLH- 39^{a} | 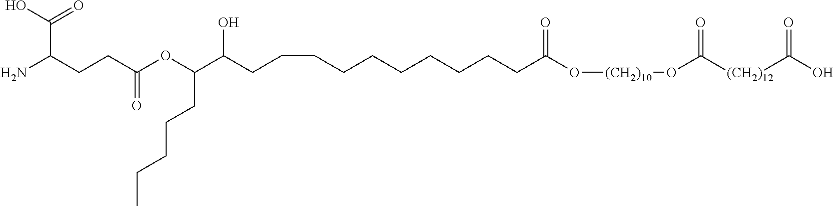 |
| GLH- 40 | 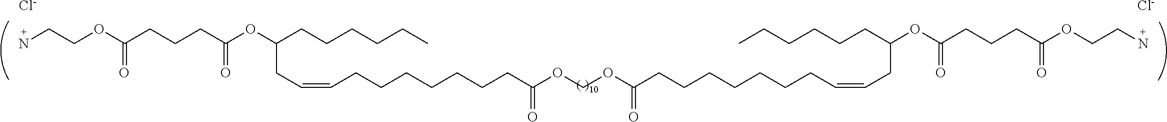 |
| GLH- 41 | 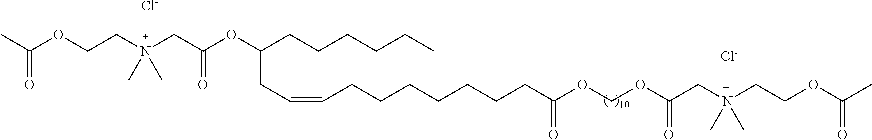 |
| GLH- 42^{a} | 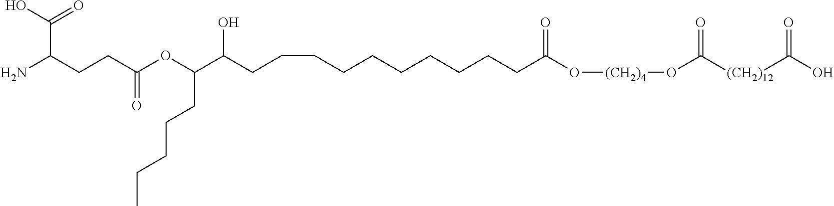 |
| GLH- 43^{a} | 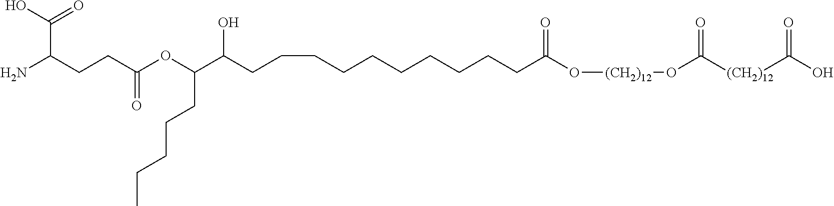 |
| GLH- 44 | 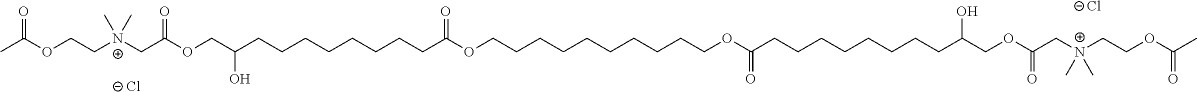 [SMILES: [Cl-] ; CC(=O)OCC[N+](C)(C)CC(=O)OCC(O)CCCCCCCCC(=O)OCCCCCCCCCCOC(=O)CCCCCCCCC(O)COC(=O)C[N+](C)(C)CCOC(C)=O ; [Cl-]] |
| GLH- 45 | 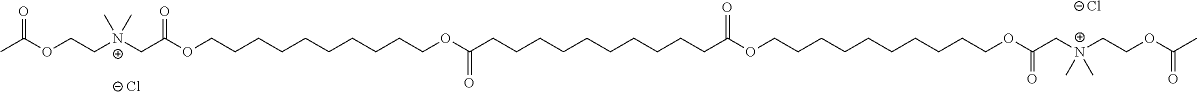 [SMILES: [Cl-] ; CC(=O)OCC[N+](C)(C)CC(=O)OCCCCCCCCCCOC(=O)CCCCCCCCCCC(=O)OCCCCCCCCCCOC(=O)C[N+](C)(C)CCOC(C)=O ; [Cl-]] |
| GLH- 46 | 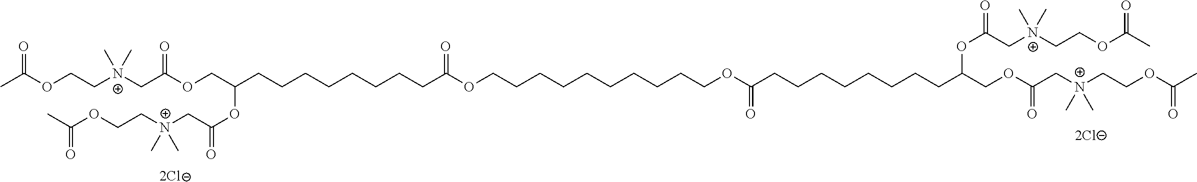 |
| GLH- 47 | 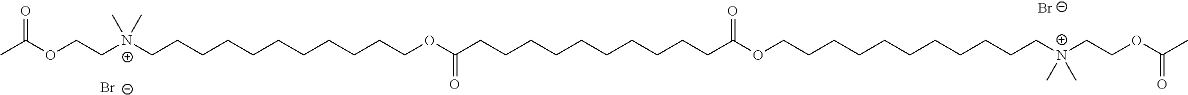 [SMILES: [Br-] ; CC(=O)OCC[N+](C)(C)CCCCCCCCCCCOC(=O)CCCCCCCCCCC(=O)OCCCCCCCCCCC[N+](C)(C)CCOC(C)=O ; [Br-]] |
| GLH- 48 | 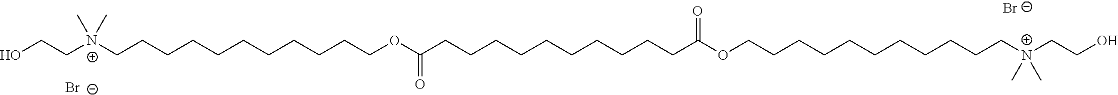 [SMILES: [Br-] ; C[N+](C)(CCO)CCCCCCCCCCCOC(=O)CCCCCCCCCCC(=O)OCCCCCCCCCCC[N+](C)(C)CCO ; [Br-]] |
| GLH- 49^{a} | 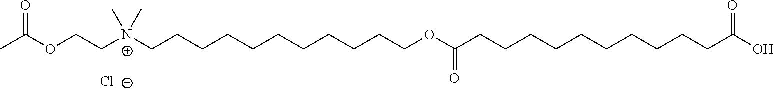 [SMILES: CC(=O)OCC[N+](C)(C)CCCCCCCCCCCOC(=O)CCCCCCCCCCC(=O)O ; [Cl-]] |
| GLH- 50^{a} | 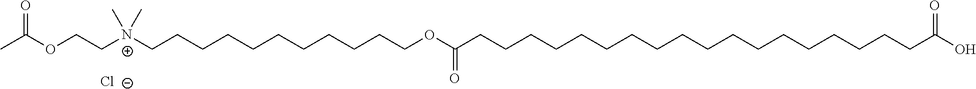 [SMILES: CC(=O)OCC[N+](C)(C)CCCCCCCCCCCOC(=O)CCCCCCCCCCCCCCCCCCC(=O)O ; [Cl-]] |
| GLH- 51^{a} | 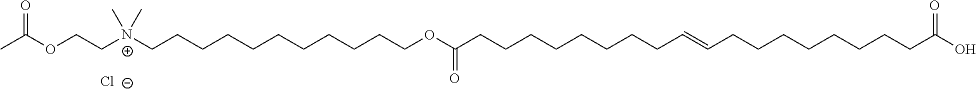 [SMILES: CC(=O)OCC[N+](C)(C)CCCCCCCCCCCOC(=O)CCCCCCCC/C=C/CCCCCCCCC(=O)O ; [Cl-]] |
| GLH- 52^{a} | 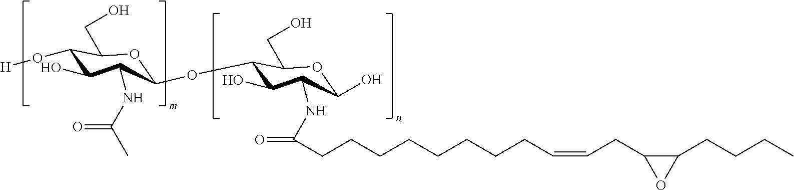 |
| GLH- 53^{a} | 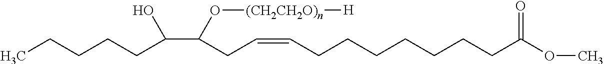 |
| GLH- 54^{a} | 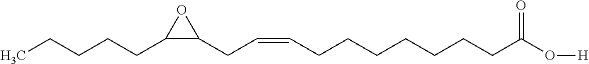 [SMILES: CCCCCC1OC1C/C=C\CCCCCCCC(=O)O] |
| GLH- 55 | 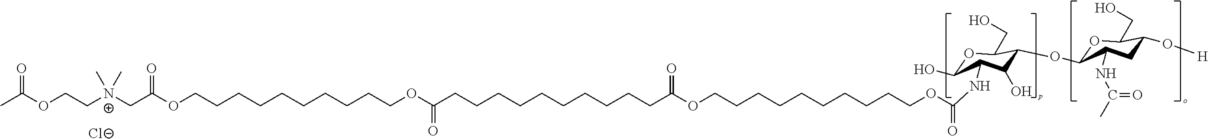 |
| GLH- 56 | 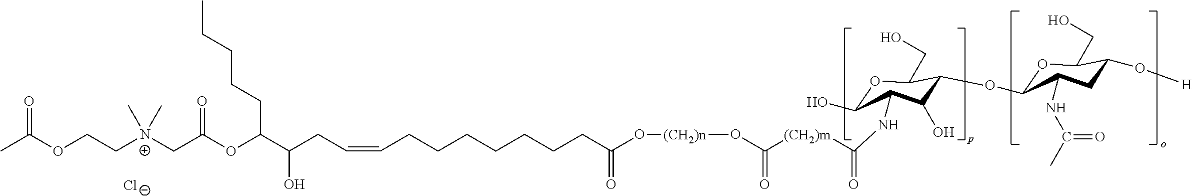 |
| GLH- 57 | 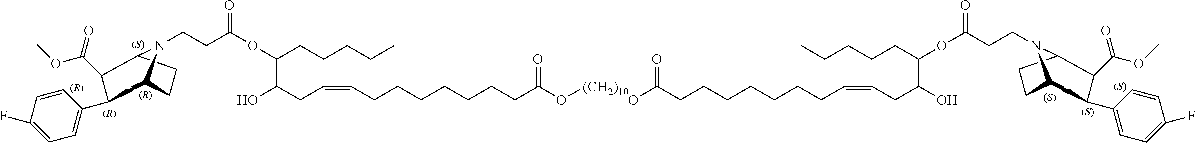 |

^{a}an intermediate

### EXAMPLE 2

### Bolavesicle Preparation and Characterization

Bolaamphiphiles, cholesterol, and CHEMS (2:1:1 mole ratio) are dissolved in chloroform or a suitable solvent. 0.5 mg of the biologically active drug dispersed in chloroform is added to the mix. The solvents are evaporated under vacuum and the resultant thin films are hydrated in 0.2 mg/mL CF solution in PBS and probe-sonicated (Vibra-Cell VCX130 sonicator, Sonics and Materials Inc., Newtown, Conn., USA) with amplitude 20%, pulse on: 15 sec, pulse off: 10 sec to achieve homogenous vesicle dispersions. Vesicle size and zeta potential were determined using a Zetasizer Nano ZS (Malvern Instruments, UK). The amount of the biologically active drug encapsulated in the vesicles can be determined by HPLC andor UV spectroscopy (G Gnanarajan, et al., 2009) after separating the non-encapsulated drug, by size exclusion chromatography (on Sephadex-G50).

### Spectral Characterization

### EXAMPLE 3

### Electron Paramagnetic Resonance (EPR)

EPR spectra of biologically active drug embedded bolavesicles resuspended in PBS can be obtained using a Bruker EMX-220 X-band (υ-9.4 GHz) EPR spectrometer equipped with an Oxford Instruments ESR 900 temperature accessories and an Agilent 53150A frequency counter. Spectra can be recorded at room temperature with the non-saturating incident microwave power 20 mW and the 100 KHz magnetic field modulation of 0.2 mT amplitude. Processing of EPR spectra, determination of spectral parameters can be done using Bruker WIN-EPR software.

### EXAMPLE 4

### Cryogenic Transmission Electron Microscopy (cryo-TEM)

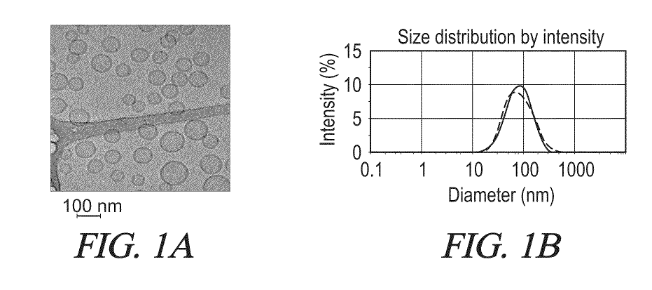

Specimens studied by cryo-TEM were prepared. Sample solutions (4 μL) are deposited on a glow discharged, 300 mesh, lacey carbon copper grids (Ted Pella, Redding, Calif., USA). The excess liquid is blotted and the specimen was vitrified in a Leica EM GP vitrification system in which the temperature and relative humidity are controlled. The samples are examined at −180° C. using a FEI Tecnai 12 G2 TWIN TEM equipped with a Gatan 626 cold stage, and the images are recorded (Gatan model 794 charge-coupled device camera) at 120 kV in low-dose mode. FIG. 1A shows TEM micrograph of vesicles from GLH-20 and their size distribution determined by DLS (FIG. 1B).

### Assays

### EXAMPLE 5

### Lipidpolydiacetylene (PDA) Assay

Lipidpolydiacetylene (PDA) vesicles (PDADMPC 3:2, mole ratio) are prepared by dissolving the lipid components in chloroform ethanol and drying together in vacuo. Vesicles are subsequently prepared in DDW by probe-sonication of the aqueous mixture at 70° C. for 3 min. The vesicle samples are then cooled at room temperature for an hour and kept at 4° C. overnight. The vesicles are then polymerized using irradiation at 254 nm for 10-20 s, with the resulting emulsions exhibiting an intense blue appearance. PDA fluorescence is measured in 96-well microplates (Greiner Bio-One GmbH, Frickenhausen, Germany) on a Fluoroscan Ascent fluorescence plate reader (Thermo Vantaa, Finland). All measurements are performed at room temperature at 485 nm excitation and 555 nm emission using LP filters with normal slits. Acquisition of data is automatically performed every 5 min for 60 min. Samples comprised 30 μL of DMPCPDA vesicles and 5 μL bolaamphiphilic vesicles assembled with biologically active drug, followed by addition of 30 μL 50 mM Tris-base buffer (pH 8.0).

A quantitative value for the increasing of the fluorescence intensity within the PDAPC-labeled vesicles is given by the fluorescence colorimetric response (% FCR), which is defined as follows²⁷:

% FCR=[(F₁−F₀)F₁₀₀]·100 Eq. 1

Where F₁ is the fluorescence emission of the lipid/PDA vesicles after addition of the tested membrane-active compounds, F₀ is the fluorescence of the control sample (without addition of the compounds), and F₁₀₀ is the fluorescence of a sample heated to produce the highest fluorescence emission of the red PDA phase minus the fluorescence of the control sample.

### EXAMPLE 6

### Cell Culture

b.End3 immortalized mouse brain capillary endothelium cells are kindly provided by Prof Philip Lazarovici (Institute for Drug Research, School of Pharmacy, The Hebrew University of Jerusalem, Israel). The b.End3 cells were cultured in DMEM medium supplemented with 10% fetal bovine serum, 2 mM L-Glutamine, 100 IU/mL penicillin and 100 μg/mL streptomycin (Biological Industries Ltd., Beit Haemek, Israel). The cells are maintained in an incubator at 37° C. in a humidified atmosphere with 5% CO₂.

### EXAMPLE 7

### Internalization of CF by the Cells In Vitro

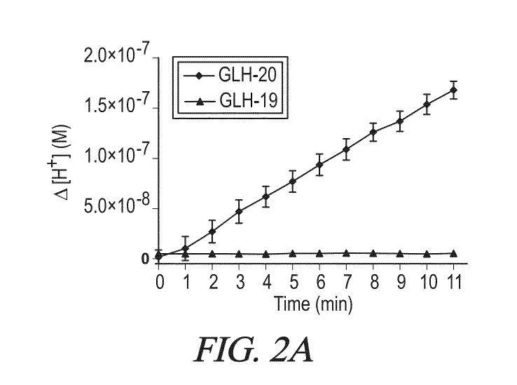

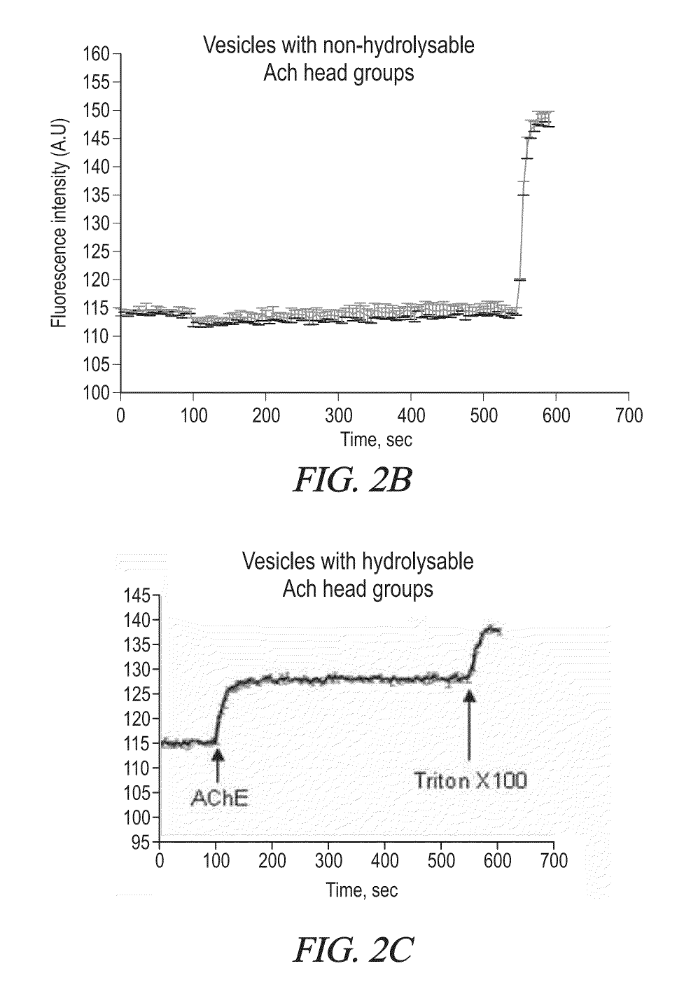

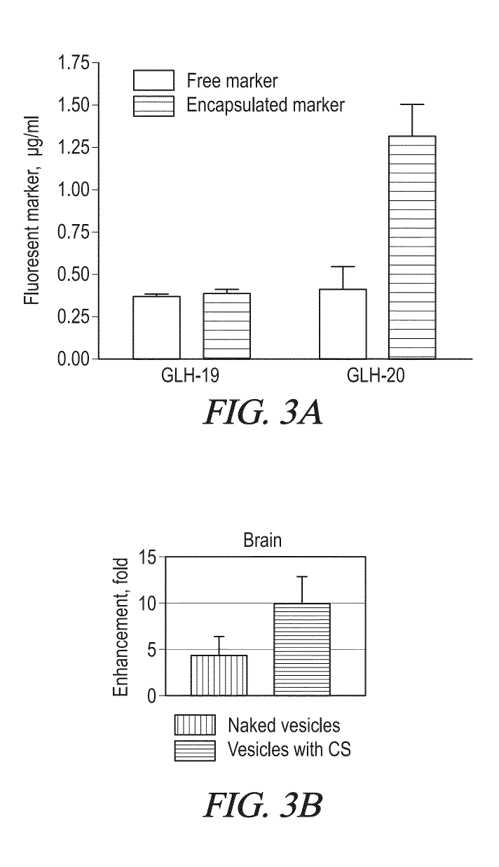

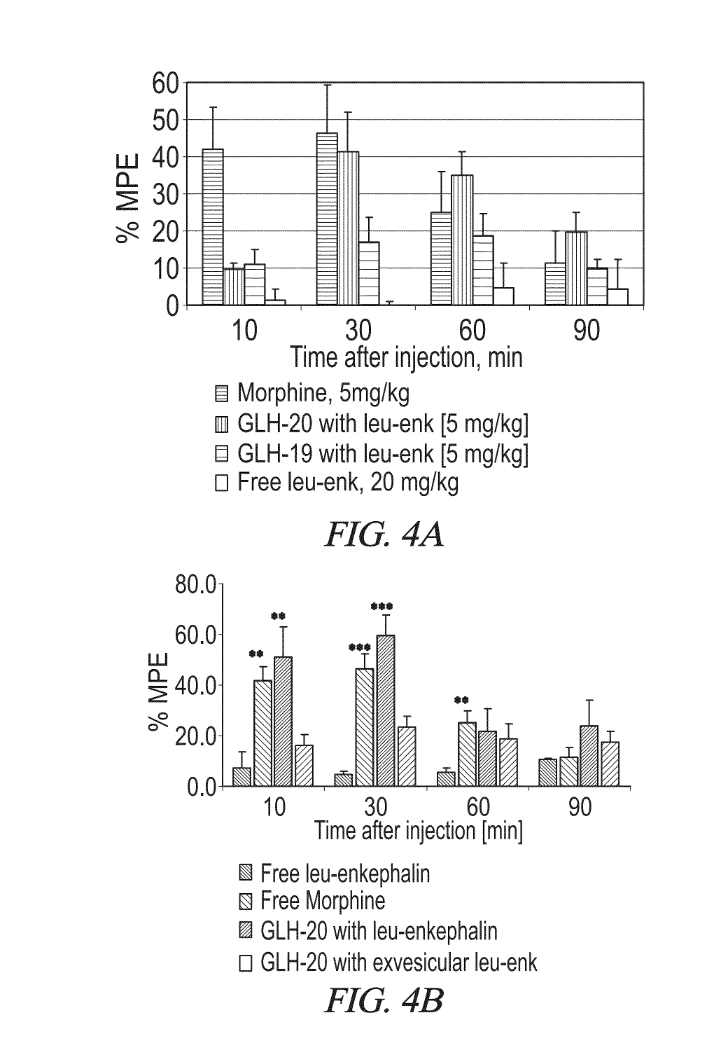

b.End3 cells are grown on 24-well plates or on coverslips (for FACS and fluorescence microscopy analysis, respectively). The medium is replaced with culture medium without serum and CF solution, or tested bolavesicles (equivalent to 0.5 μg/mL CF), or equivalent volume of the medium are added to the cells and incubated for 5 hr at 4° C. or at 37° C. At the end of the incubation, cells are extensively washed with complete medium and with PBS, and are either detached from the plates using trypsin-EDTA solution (Biological Industries Ltd., Beit Haemek, Israel) and analyzed by FACS (FACSCalibur Flow Cytometer, BD Biosciences, USA), or fixed with 2.5% formaldehyde in PBS, washed twice with PBS, mounted on slides using Mowiol-based mounting solution and analyzed using a FV1000-IX81 confocal microscope (Olympus, Tokyo, Japan) equipped with 60× objective. All the images are acquired using the same imaging settings and are not corrected or modified. FIGS. 2A-2C shows head group hydrolysis by AChE (FIG. 2A) of GLH-19 (blue) and GLH-20 (red) and release of CF from GLH-19 vesicles (FIG. 2B) and GLH-20 vesicles (FIG. 2C). AChE causes the release of encapsulated material from GLH-20 vesicles, but not from GLH-19 vesicles (FIG. 2A). The vesicles are capable of delivering small molecules, such as carboxyfluorescein (CF), into a mouse brain, but the fluorescent dye accumulates only if it is delivered in vesicles that release their encapsulated CF in presence of AChE, namely, GLH-20 vesicles (FIG. 3A). These results suggest that the release is due to head group hydrolysis by AChE in the brain. Corroboration for this conclusion also comes from an experiment showing that when an analgesic peptide is delivered to the brain by the bola vesicles, analgesia (which is caused when the encapsulated peptide is released in the brain) was observed only with GLH-20 vesicles, but not by GLH-19 vesicles (FIG. 4A).The vesicles do not break the BBB, but rather penetrate it in their intact form, as indicated by the finding that analgesia is obtained only when enkephalin is administered while encapsulated within the vesicles, but not when free enkephalin is administered together with empty vesicles (FIG. 4B).

The ACh head groups also provide the vesicles with cationic surfaces, which promote penetration through the BBB [Lu et al, 2005] and transport of the encapsulated material into the brain. Toxicity studies showed that the dose which induced the first toxic signs was 10-20 times higher than the doses needed to obtain analgesia by encapsulated analgesic peptides.

The addition of chitosan (CS) surface groups, by employing CS-vernolate conjugates, increased BBB permeability of the vesicles (FIG. 4B), probably by increasing transcytosis [Newton, 2006]. However, the CS groups, when added to the vesicles by employing fatty acid-CS conjugate (in this case, vernolic acid), are not stable in circulation as surface groups because of the low energy barrier for lipid exchange of such conjugates. The inventors propose to make stabilized CS surface groups by using bolas that the inventors will synthesize with covalently-attached CS head groups [see, Experimental Design and Methods, below].

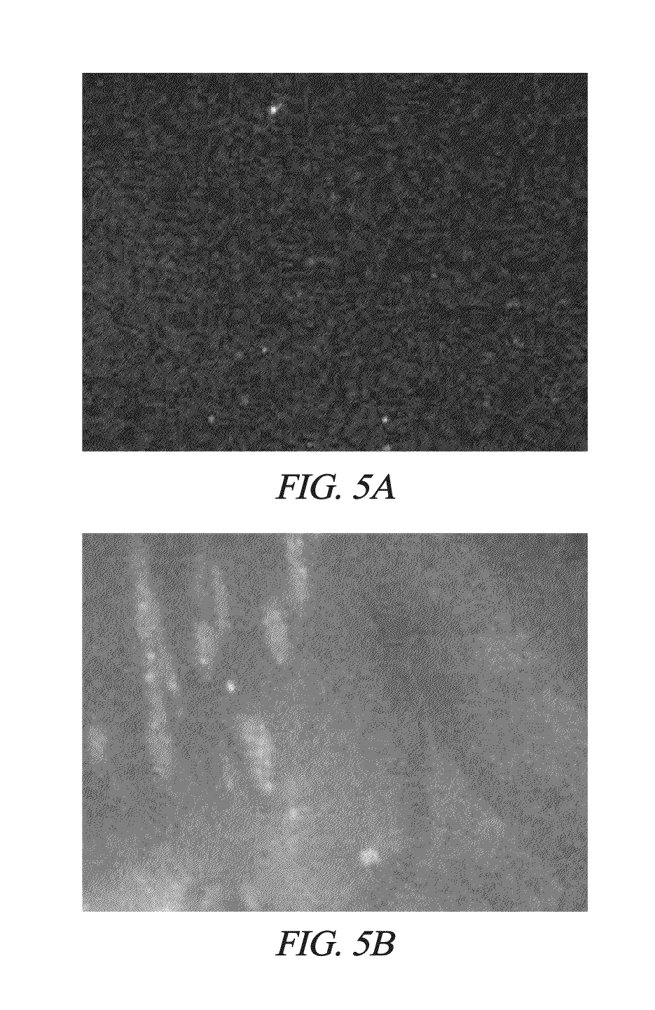

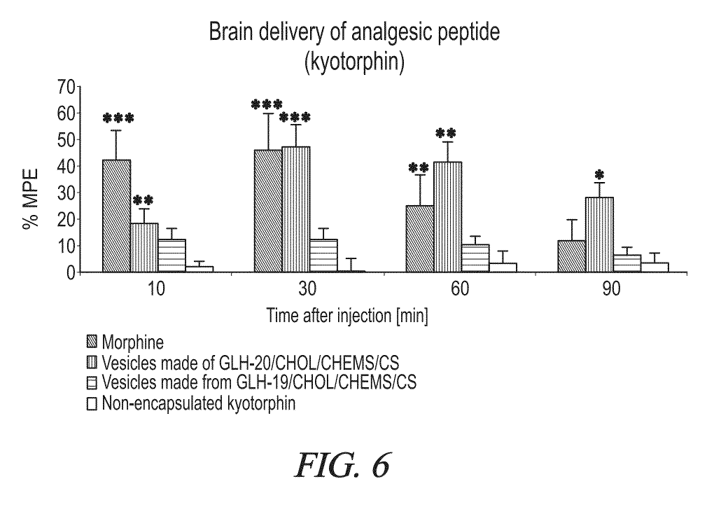

In addition to the peptide leu-enkephalin, and the small molecules: CF, uranyl acetate, kyotorphin and sucrose, the inventors have also successfully encapsulated in these vesicles the proteins albumin and trypsinogen and the polysaccharide Dextran-FITC (MW 9000). Albumin-FITC, encapsulated, was delivered successfully to the brain (FIG. 5B), while un-encapsulated albumin-FITC showed little, if any, brain accumulation (FIG. 5A), indicating that the vesicle transported the protein into the brain through the BBB. These results strongly suggest that the vesicles can be made to encapsulate other molecules, such as anti-retroviral drugs, and deliver them into the brain without harming the BBB.

### EXAMPLE 8

### Statistical Analysis

The data are presented as mean and standard deviations (SD) or standard errors of mean (SEM). Statistical differences between the control and the studied formulations are analyzed using ANOVA followed by Dunnett post-test using InStat 3.0 software (GraphPad Software Inc., La Jolla, Calif., USA). P values of less than 0.05 are defined as statistically significant.

### EXAMPLE 9

### Encapsulation of CPT-11

A) Optimization of vesicle formation: Vesicles are prepared by film hydration, followed by sonication. Each of the vesicle formulations can be examined for vesicle size (by dynamic light scattering), morphology (by cryo-transmission electron microscopy), zeta potential (by Zeta Potential Analyzer) and stability (by fluorescence measurements of encapsulated CF at various times after vesicle preparation). Stability of vesicles can be determined in presence and absence of ChE, with and without an inhibitor of the enzyme (e.g., pyridostigmine)

B) Encapsulation of CPT-11: To successfully encapsulate CPT-11 (MW 586.67, water solubility of 25 mg/ml with bis-piperidine moiety, which forms an ammonium salt in acid) within the vesicles, the active loading approach can be used. CPT-11 can be encapsulated in its active lactonic form, and not in the inactive carboxylate form. The loading conditions based on conditions developed for liposomal formulations using a pH gradient between the liposome core can be used and the bathing medium, whereas the internal volume can be acidic compared to the external solution.

For encapsulations, vesicles can be formed in acidic buffers, such as citrates. The vesicles can be purified on a GPC column to separate encapsulated CPT-11 from non-encapsulated material. Percent encapsulation can be determined by UV absorption of the CTP-11's aromatic groups after lysis with a detergent. To maximize CPT-11 loading and minimize leakage, the composition of the vesicle's membrane can be optimized by varying both the ratio between bolaamphiphiles in the vesicle formulation and the proportion of different additives used in the vesicle formulation, such as cholesterol hemisuccinate and neutral cholesterol; or drug-loading with respect to the relative concentration of CPT-11 to vesicles, the temperature during loading, internal buffer composition and the pH gradient across the vesicle's membrane.

The entrapped CPT-11 may be stabilized by adding, to the vesicle core, agents that help to prevent leakage, such as dextran sulfate28, copper sulfate and other transition metal salts29, and polymeric or highly charged nonpolymeric polyanionic trapping agents.

### EXAMPLE 10

### Determination of Encapsulated CPT-11 Activity

To ensure that the encapsulation process did not reduce the cytotoxic activity of CPT-11, the encapsulated CPT-11 can be released from the vesicles by ChE treatment, and the released CPT-11 can be collected from the supernatant following centrifugation. The IC₅₀ of the released CPT-11 can be determined by using U87 glioblastoma cell line and by a standard viability assay (e.g., MTT) in comparison to that of standard CPT-11.

### EXAMPLE 11

### Synthesis of Bolaamphiphiles From Jojoba Oil

This example describes the synthesis of three new, illustrative, bolaamphiphiles from jojoba oil, which are designated GLH-58, GLH-59, and GLH-60, and are depicted below.

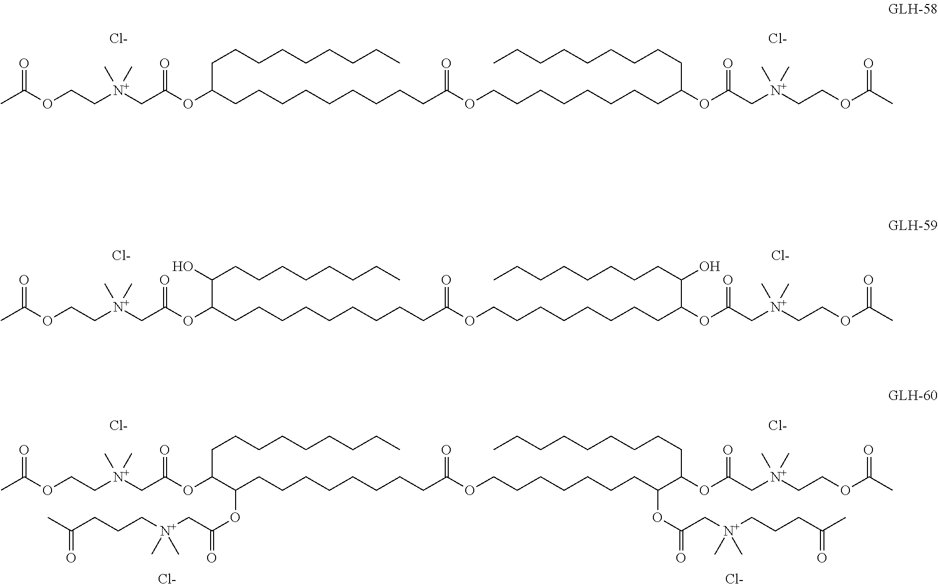

We have described novel bolaamphiphiles with acetylcholine (ACh) head groups and shown that these bolaamphiphiles interact with small interference RNA molecules (siRNA) and form particles that are internalized by cells and silence genes following their internalization both in vitro and in vivo. These studies indicated that the ACh head groups play a major role in the interactions between the siRNA and the bolaamphiphile and additions of head groups may increase the amount of the siRNA that binds the bolaamphiphile. The present disclosure describes the synthesis of a bolaamphiphile with more than two ACh head groups and the investigation thereof with respect to their interactions with siRNA.

The bolaamphiphiles described in previous sections were synthesized from fatty acids derived from triglyceride vegetable oils (i.e. vernolic and oleic acids). This is a multistage synthesis, since when fatty acids derived from triglyceride oils are used as the starting material for the synthesis of bolaamphiphiles, the skeleton of the bolaamphiphile has to be synthesized first and only then, the ACh head groups are attached to the bolaamphiphilic skeleton.

In order to simplify the synthesis of boloaamphiphiles with ACh head groups, particularly bolaamphiphiles with more than two ACh head groups, we used jojoba oil as the starting material

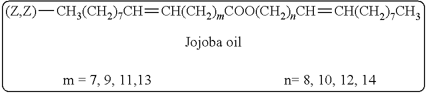

In contrast to the triglyceride vegetable oils, jojoba oil is a liquid wax with a 40-42 carbon atom chain composed mainly of straight chain monoesters of C₂₀ and C₂₂ monounsaturated acids and alcohols. Jojoba oil constitutes a unique starting material for the synthesis of bolaamphiphiles as its chemical structure may provide a hydrophobic skeleton of 40-44 carbon atoms and the ACh head groups can be bound directly to the jojoba oil, which is used as the bolaamphiphilic skeleton.

The two double bonds on either side of the jojoba's aliphatic chain are used to attach the head groups. The ACh head groups can be attached to the jojoba skeleton in two different ways: (a) direct addition of haloacetic acid to the double bond followed by quaternization of the head group, or (b) epoxidation of the double bonds and opening the epoxy group; e.g. esterification of the hydroxyl groups formed with a haloacetic acid followed by quaternization of the tertiary amine to give a bolaamphiphilic compound. Two examples are provided in the following structures:

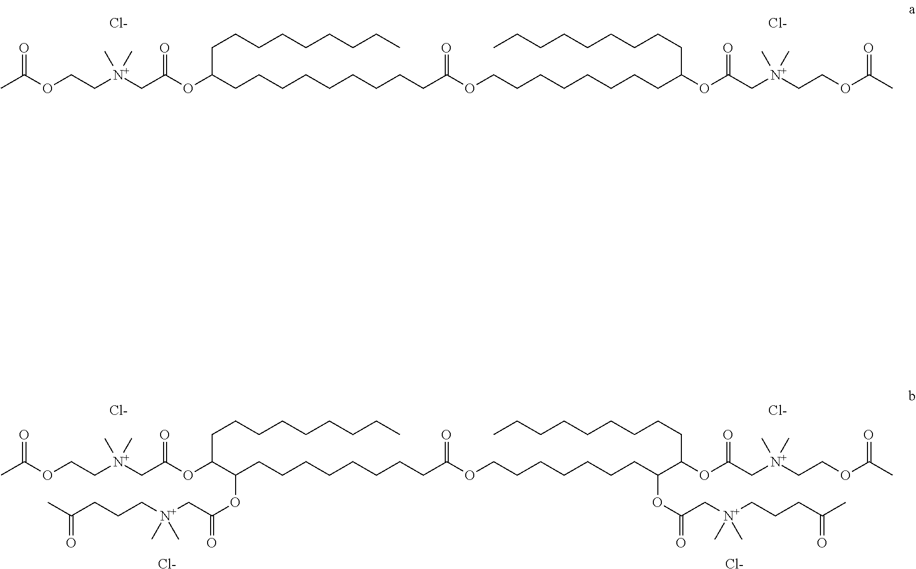

The chemical structure of bolaamphiphilic compounds with ACh head groups that were synthesized from jojoba oil include the above structures, wher compound (a) is designated as GLH-58, a bolaamphiphile with two ACh head groups, and compound (b) is designated GLH-60, a bolaamphiphile with four ACh head groups.

### EXAMPLE 12

### Synthesis of the Bolaamphiphilic Compound, GLH-58

In this Example, the bolaamphiphilic compound, GLH-58 was synthesized through a direct addition of a halo acetic acid to the double bonds of jojoba oil. A first step involved synthesis of the dichloroacetate derivative of jojoba oil. In one embodiment, the method described by Carey [Carey F. A., Sundberg R. J. Advanced Organic Chemistry fifth edition, Part A: Structure and Mechanisms. Chapter 5. Polar Addition and Elimination Reactions (2008): 473-477] for a direct addition of chloroacetic acid to double bonds was employed. However, the addition of chloroacetic acid to jojoba oil under these conditions (without using a catalyst) did not result in the formation of a product. Therefore, the reaction has been performed under acidic conditions, in the presence of a concentrated H₂SO₄, or in the presence of a cation exchange resin [Patwardhan A. A, Sharma M. M., Esterification of Carboxylic Acids with Olefins using Cation Exchange Resins as Catalysts. Reactive Polymers, 13 (1990): 161-176, and Chakrabarti A., Sharma M. Esterification of Acetic Acid with Styrene: Ion Exchange Resins as Catalysts. Reactive Polymers, 16 (19911992): 51-59]. We found that Jojoba oil (compound 1 in Scheme 1, below) reacted with a threefold excess of chloroacetic acid (compound 2 in Scheme 1) at 90° C. in the presence of the catalyst Amberlyst 15, which was dried by toluene azeotropic distillation.

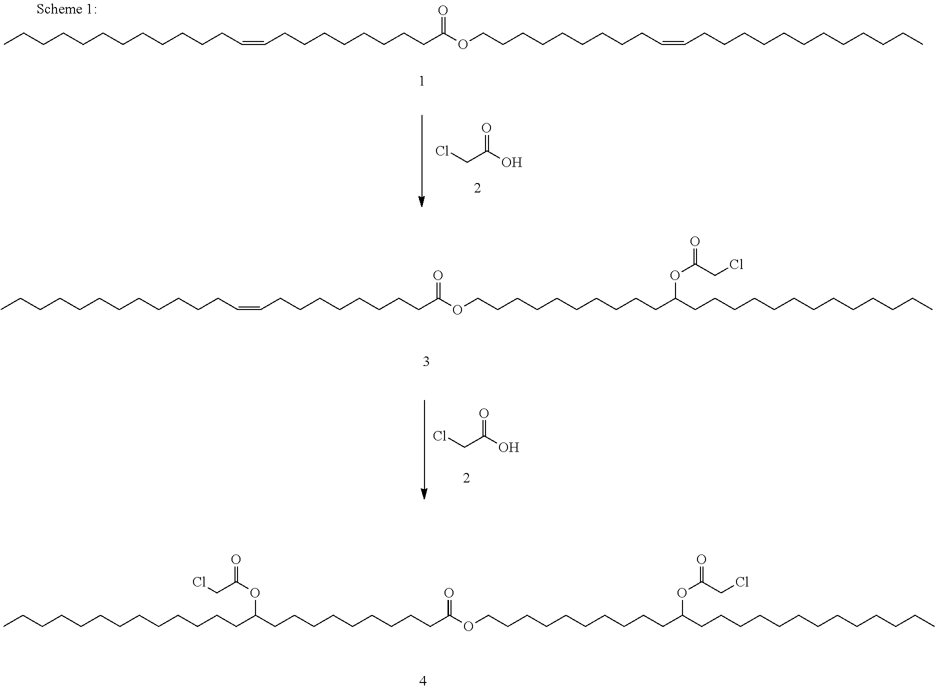

The progress of the reaction was followed by monitoring the products on TLC and HPLC. The appearance of two new products in addition to the starting material was observed after about two hours. The two products were isolated by a flash column chromatography and identified as jojoba monochloroacetate (compound 3 in Scheme 1) and jojoba dichloroacetate (compound 4 in Scheme 1).

The FT-IR spectrum of the jojoba monochloroacetate 3 showed the peaks characteristic of a double bond at 3006 cm−1, of a carbonylic ester group at 1737 cm−1, and a new chloroacetate ester group at 1759, 1289 and 1254 cm−1. By comparison, the FT-IR spectrum of jojoba dichloroacetate 4 showed the disappearance of the absorption bands characteristic to the double bond and the appearance of the new absorption band for the new chloroacetate ester groups, very similar to those of the jojoba monochloroacetate 3. The ratio of the peak area of the chloroacetate (1758 cm-1)to the peak area of the original ester group of jojoba (1735 cm−1) in FT-IR was found to be equal to 0.3 for the monochloroacetate 3 and 0.6 for the dichloroacetate 4.

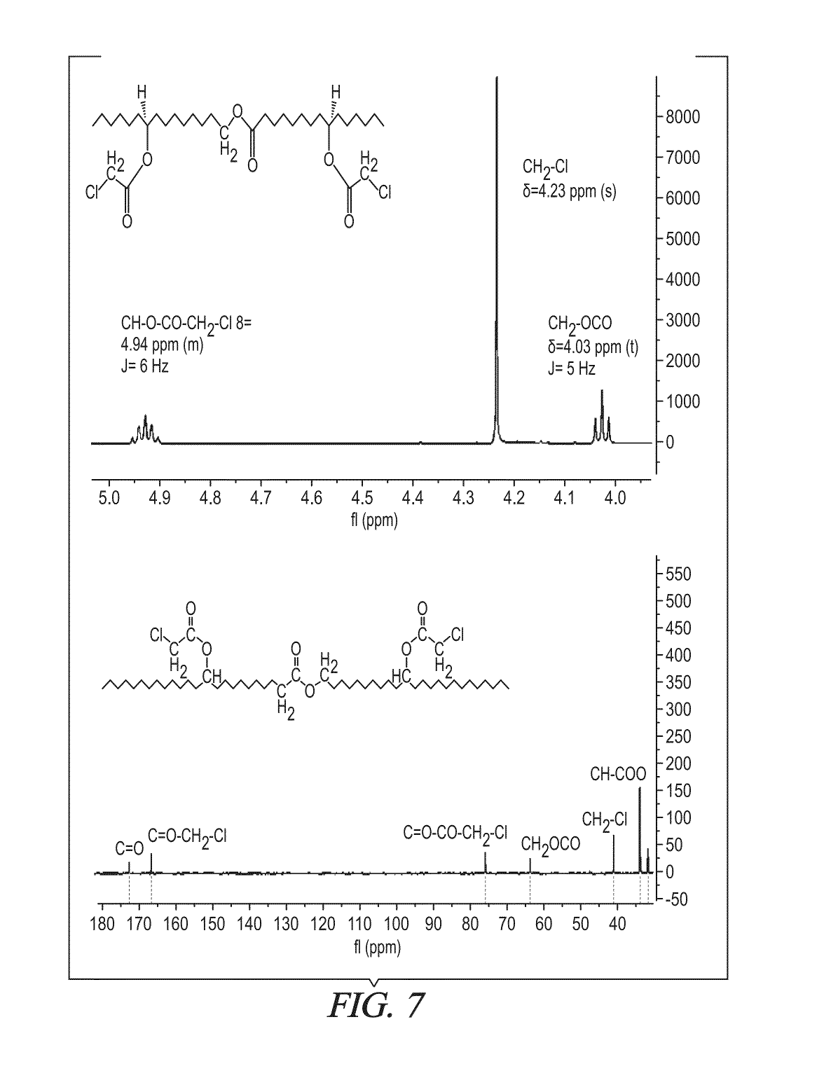

The NMR spectrum of jojoba dichloroacetate 4 showed the disappearance of the double bonds at 5.2 ppm. The new chemical shifts characteristic of the CH moiety of the new ester group: CH—O—CO—CH₂—Cl appeared as a quintet at 4.94 ppm in ¹H-NMR and at 75.9 ppm in ¹³C-NMR; the chloromethylene group CH—O—CO—CH₂—Cl as singlet at 4.23 ppm and at 41.00 ppm, correspondingly, and the new carbonyl group: C═O—CH₂—Cl at 166.88 ppm (FIG. 7).

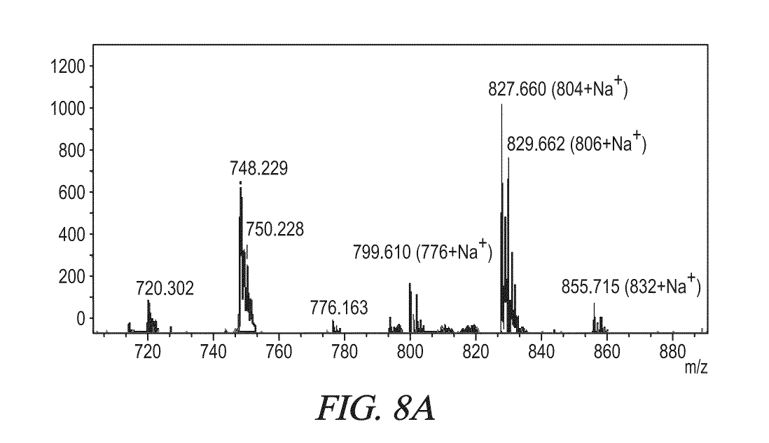

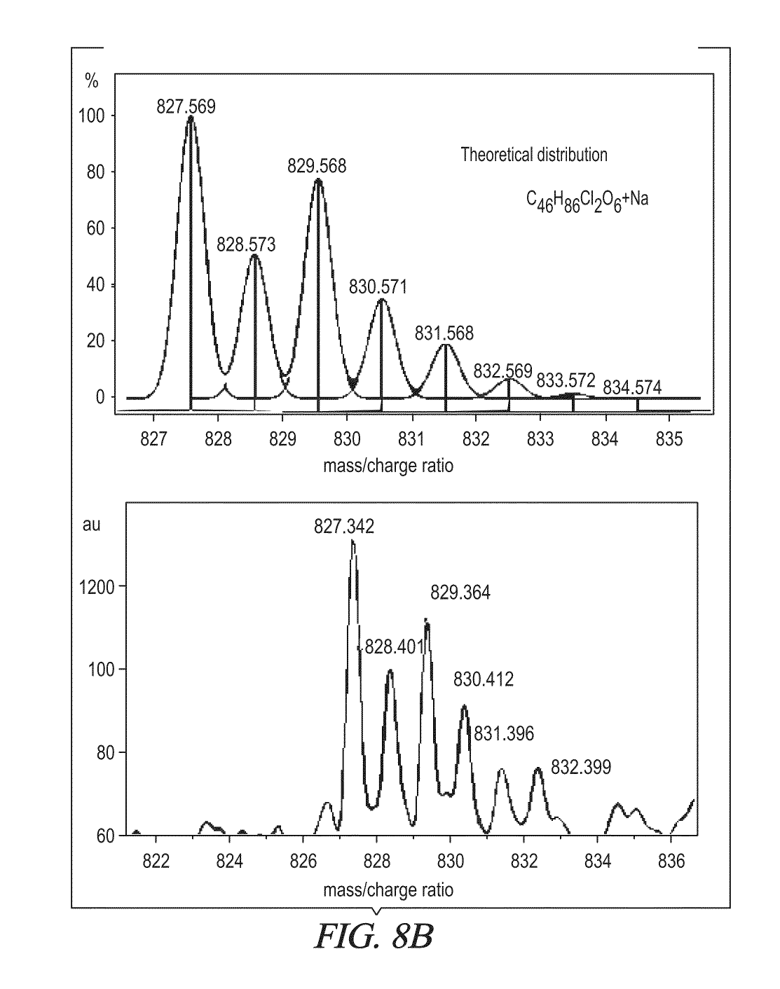

The HPLC chromatogram of the products showed five main peaks, indicating on 5 components of the jojoba dichloroacetate derivatives. The different components of the reaction mixture were identified by MALDI-MS (FIG. 8A) as the jojoba dichloroacetate derivatives 4 (C₄₈H₉₀Cl₂O₆, C₄₆H₈₆Cl₂O₆, C₄₄H₈₂Cl₂O₆, C₄₂H₇₈Cl₂O₆, C₄₀H₇₄Cl₂O₆). The ratio of the isotopes in the main compound of this mixture (C₄₆H₈₆Cl₂O₆⁺Na) was consistent with the calculated value (FIG. 8B). The abundance of dichloroacetate derivatives corresponds to the abundance of original jojoba oil molecules.

Synthesis of bolaamphiphile GLH-58: In the last stage of the synthesis, the jojoba oil dichloroacetate 4 was used as the alkylating agent for the quaternization of the tertiary amine N,N′-dimethylaminoethyl acetate (compound 5 in Scheme 2). The Jojoba dichloroacetate 4 was reacted with an excess of the amine 5 at 60° C. for 5 h to obtain the bolaamphiphile GLH-58 that contains two ACh head groups as depicted in Scheme 2:

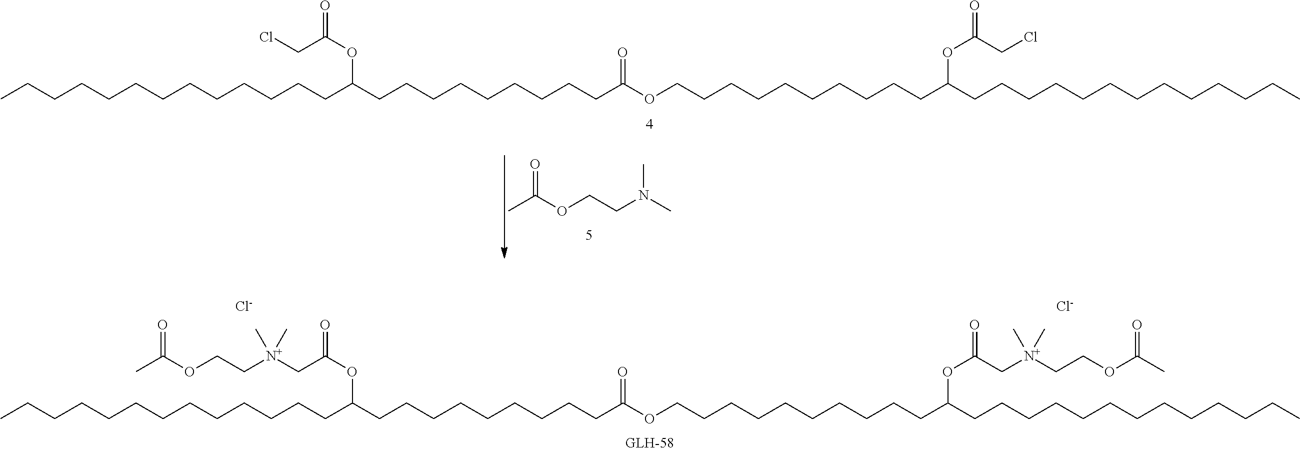

TLC of the reaction mixture showed a new compound already after 2 h, and after 5 h all the dichloroacetate derivatives 4 were consumed. The reaction mixture was cooled to room temperature; hexane was added to remove the excess of the amine 5. The hexane extraction process was repeated several times. The lower phase, containing the crude product, was collected and the solvent was removed under reduced pressure and further purified by flash chromatography using acetonitrile:water (10:1) as the eluent. The purity of the GLH-58 was 98.4%, as determined by argentometric titration and its appearance was viscous liquid.

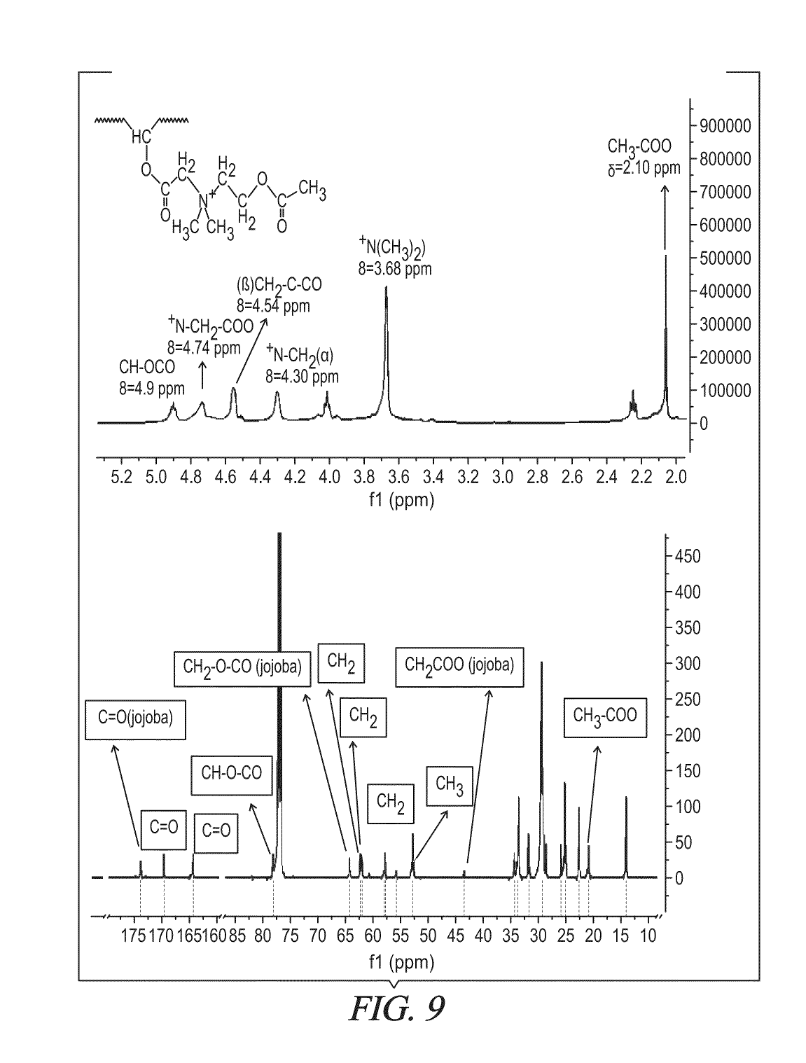

In the FT-IR spectra, the absorption bands, characteristic of the chloroacetate ester, at 1757, 1290, 1257, and 1184 cm−1 and of the CCl bond at 784 cm⁻¹ disappeared, and new absorption bands appeared at 3383 cm⁻¹, 3017 cm−1 and are attributed to the C—H stretch of the nitrogen-bound methyl groups. The chemical shifts characteristic of the chloromethylene group (—CH₂—Cl) of intermediate 4 at 4.23 ppm and 41.00 ppm in the ¹H- and ¹³C-NMR, respectively, disappeared and new signals of the quaternary ammonium group appeared (FIG. 9). The chemical shifts of the methyl groups of the quaternary nitrogen (CH₃)₂N⁺ appeared at 3.68 and 52.78 ppm in the ¹H-NMR and ¹³C-NMR spectra, respectively, and for CHO—C═O at 4.90 ppm and 78.26 ppm, respectively. The chemical shifts for the methyl of the ACh group CH₃—C(O)—O appeared at 2.10 and 20.88 ppm, and for the carbonyl carbon CH₃—C(O)—O, at 169.88 ppm (FIG. 9).

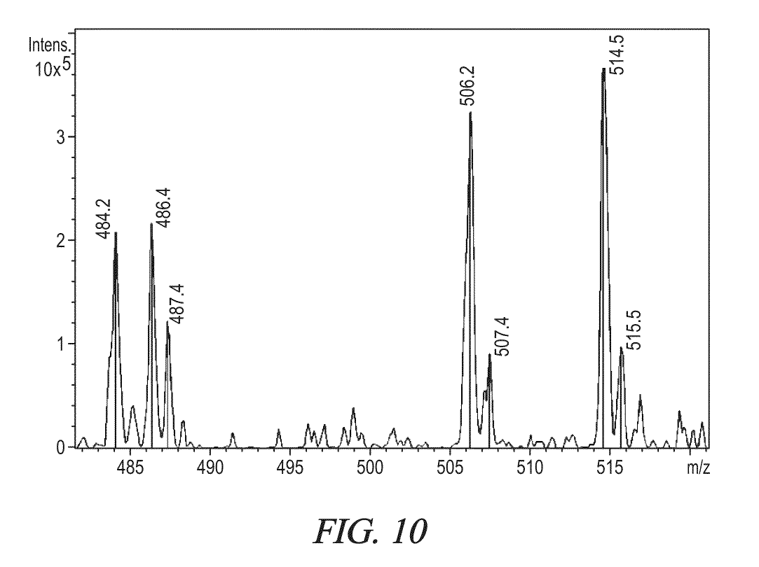

The MS spectrum of GLH-58 (FIG. 10) showed the presence of three main peaks for the quaternary bolaamphiphile [M-2Cl/2]⁺: 486.4, 506.2 and 514.5 for C₅₈H₁₁₂O₁₈N₂Cl₂, C₅₉H₁₁₄O₁₀N₂Cl₂ and C₆₀H₁₁₆O₁₀N₂Cl₂, respectively. These results are consistent with the theoretical molecular mass of the bolaamphiphile with the two ACh head groups derived from the corresponding jojoba dichloroacetate main molecules.

### EXAMPLE 13

### Synthesis of Bolaamphiphile GLH-60 Through Epoxidation of Jojoba Oil

GLH-60, a bolaamphiphilic compound with four ACh head groups, was synthesized as depicted in Scheme 3, below, using jojoba diepoxide 7 as the starting material.

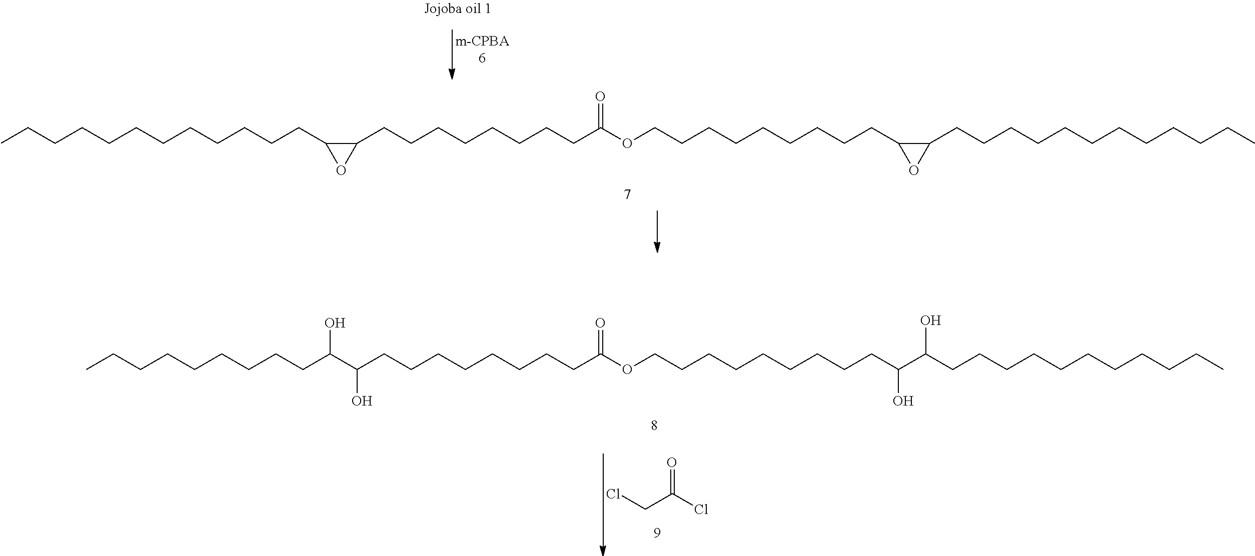

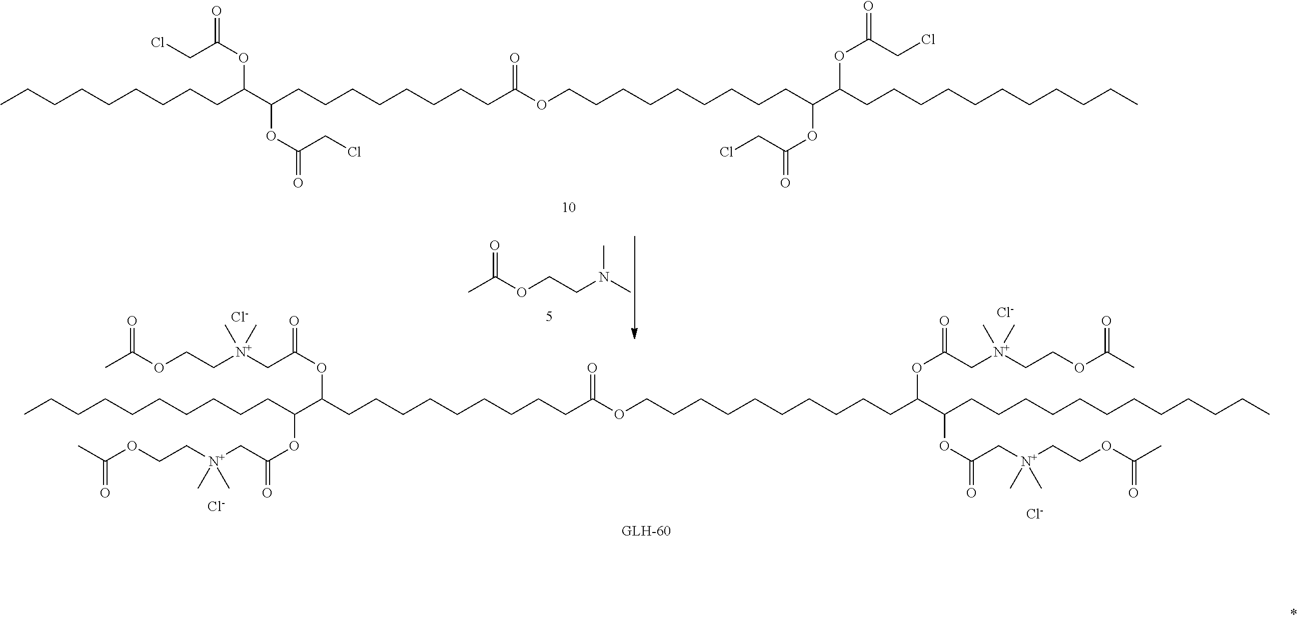

Synthesis of jojoba diepoxide 7: The epoxidation of jojoba oil was carried out using an excess of m-chloroperbenzoic acid (m-CPBA)—compound 6 in Scheme 3 [Lynch B. M. and Pausacker K. H., J. Chem. Soc., (1955): 1525; Kim C. C., Traylor T. G, and Perrin. C. L. MCPBA Epoxidation of Alkenes: Reinvestigation of Correlation between Rate and Ionization Potential. J. Am. Chem. Soc. 120 (1998): 9513-9516; Eugeniuzs M., Smagowicz A., Lewandowski G. Optimization of the Epoxidation of Rapeseed oil with Peracetic Acid. Organic Process Research & Development (2010): 1094-1101]. The reaction was performed in CHCl₃ at 5-10° C. and monitored by thin layer chromatography (TLC). After two hours the total disappearance of the double bond, characteristic of jojoba oil, and the appearance of a new polar compound was observed. The jojoba diepoxide (compound 7 in Scheme 3) was obtained in a 74.6% yield and 84.5% purity as determined by potentiometric titration.

The FT-IR of jojoba diepoxide 7 showed the typical epoxy group absorption bands at 820 and 842 cm⁻¹ and the disappearance of the absorption peak at 3004 cm⁻¹ the C—H stretching in the double bond.

In the NMR spectrum, the peak of the double bond of jojoba oil disappeared and a new signal, characteristic of the epoxy group protons, appeared at 2.77 ppm and 57.29 ppm in the ¹H- and ¹³C-NMR spectra, respectively.

Synthesis of tetrahydroxy jojoba oil: The hydrolysis of epoxides is pH dependent and can occur through acid, neutral or base promoted processes, but the acid and neutral processes dominate over environmentally significant pH ranges [Rogers E. Harry-O'kurua, Abdellatif Mohamedb, Thomas P. Abbott. Synthesis and Characterization of Tetrahydroxy Jojoba Wax and Ferulates of Jojoba Oil. Science 22 (2005): 125-133]. The hydrolysis of jojoba diepoxide by the opening of the epoxy groups to form a diol on each side of the ester (scheme 3) was carried out in the presence of concd. H₂SO₄. After washing and precipitation of the product with petroleum ether, the tetrahydroxy jojoba oil 8 was obtained as a white powder in 81% yield.

The FT-IR spectrum of intermediate 8, the tetrahydroxy jojoba oil, showed absorption bands characteristic of the —OH at 3310 cm⁻¹ and for C—O—C at 1183 cm⁻¹. In ¹H-NMR and ¹³C-NMR was observed the CH—OH group at 3.37 and 74.40 ppm respectively.

Synthesis of tetrachloroacetate of jojoba oil 10: The esterification of tetrahydroxy jojoba oil (compound 8 in Scheme 3) was performed by using an excess of chloroacetyl chloride (compound 9 in Scheme 3), in chloroform as the solvent at 0° C. in the presence of pyridine (scheme 3). The tetrachloroacetate of jojoba oil (compound 10 in Scheme 3) was separated from the reaction mixture by flash column chromatography, using chloroform as the eluting solvent, and appeared as a yellow semi-solid which was obtained in 59% yield.

FT-IR spectra of compound 10 showed that the absorption bands, characteristic of the hydroxyl groups, disappeared and new absorption bands, characteristic of the chloroacetate group, appeared at 1762 (C═O) and 1286 cm⁻¹ (C—O).

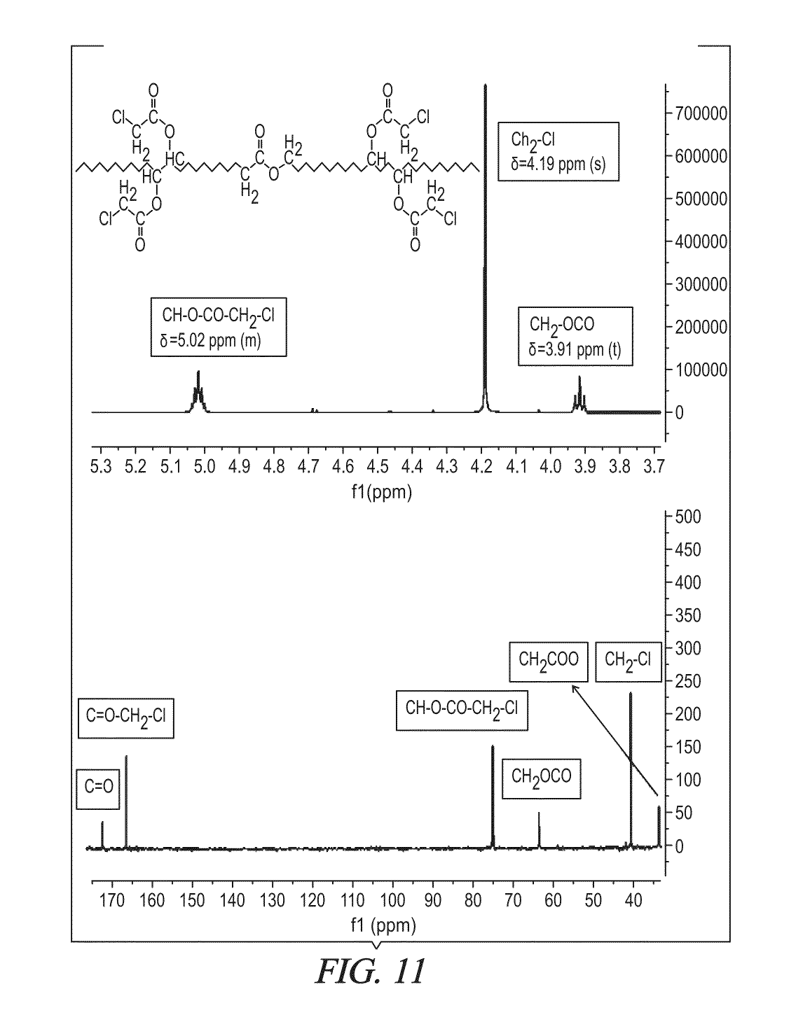

The NMR analysis showed new chemical shifts, characteristics of the methane proton CH—O—CO—CH₂—Cl, as multiplet at 5.02 ppm and at 75.11 ppm, in 1H- and 13C-NMR, respectively. The chloromethylene group CH—O—CO—CH₂—Cl appeared as a singlet at 4.19 ppm and at 40.68 ppm in ¹H- and ¹³C-NMR, respectively and the new carbonyl group C═O—CH₂—Cl at 166.79 ppm (FIG. 11).

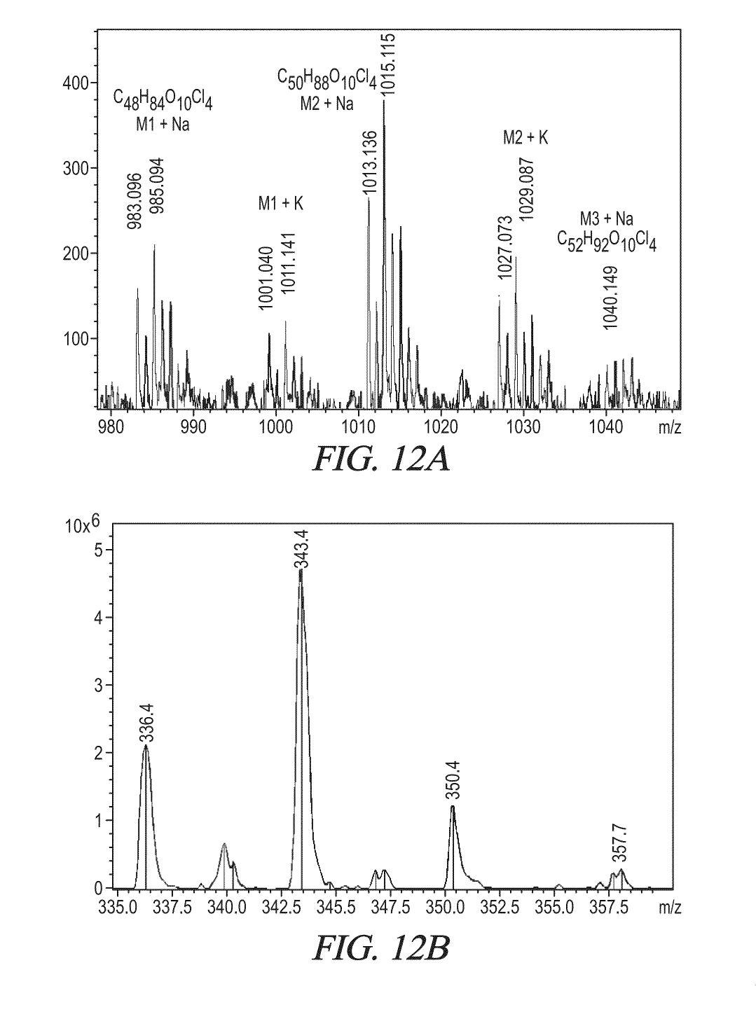

The MALDI-MS of compound 10, C₅₀H₈₈O₁₀C₁₄ and C₄₈H₈₄O₁₀C₁₄ (FIG. 12) is consistent with the theoretical molecular mass of tetrachloroacetate of jojoba oil 10 derived from the esters with 42 carbons and 40 carbons. The isotope abundance pattern for each molecular weight corresponds for a molecule containing four chlorine atoms.

Synthesis of bolaamphiphile GLH-60: In the last stage of the synthesis the tetrachloroacetate intermediate 10 was reacted with a small excess of N,N′-dimethylaminoethyl acetate 5 at 60° C. for 6 h to obtain the bolaamphiphile GLH-60 with four ACh head groups (Scheme 3). The non-reacted N,N′-dimethylaminoethyl acetate was separated from the crude product by adding hexane followed by decantation as was described above. The bolaamphiphilic compound, GLH-60, was obtained as a viscous liquid with a purity of 96%, as determined by argentometric titration.

The MALDI MS of GLH-60: m/z [M-4Cl/4]⁺: 336.4, 342.4, 350.4 and 357.4 for C₇₂H₁₃₆O₁₈N₄Cl₄, C₇₄H₁₄₀O₁₈N₄Cl₄, C₇₆H₁₄₄O₁₈N₄Cl₄, C₇₈H₁₄₈O₁₈N₄Cl₄ was consistent with the theoretical molecular mass of a bolaamphiphile with the four ACh head groups derived from the corresponding esters of jojoba oil (FIG. 12). Jojoba Tetrachloroacetate is compound 10 in Scheme 3.

As described here a novel formulations of bolavesicles can be produced through co-assembly of biologically active drugs with bolaamphiphilelipid unilamellar vesicles. The formulations can be examined for their chemical and biophysical properties.

The incorporation of biologically active drug within the bolavesicles is shown to significantly modulate interactions with membrane bilayers in model systems. This observation is important, suggesting that biologically active drugs encapsulated in bolavesicles might be excellent candidates for targeting and transport of different molecular cargoes into the brain.

From the foregoing description, various modifications and changes in the compositions and methods provided herein will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

Chemical structures shown herein were prepared using ISIS DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

### REFERENCES

*Abu Hammad I, Popov M, Linder C, Grinberg S, Heldman E, Stepensky D (2011) Bolaamphiphilic nanovesicles for the delivery of proteins to the brain, submitted to the Journal of Controlled Release.
Agyare, E K, Kandimalla K K, Poduslo J F, Yu C C, Ramakrishnan M, Curran G L (2008) Development of a smart nano-vesicle to target cerebrovascular amyloid deposits and brain parenchymal plaques observed in Alzheimer's disease and cerebral amyloid angiopathy. Pharm Res November; 25(11):2674-2684.
Fuhrhop J. H. and Wang T. (2004) Bolaamphiphiles, Chem. Rev. 104:2901-2937.
Gisslen M and Hagberg L and Hagberg (2001) Antiretroviral treatment of central nervous system HW-1infection: a Review. HIV Medicine (2001) 2, 97-104.
G Gnanarajan, A K Gupta, V Juval, P Kumar, P K Yadav, P Kailash “A validated method for development of tenofovir as API and tablet dosage forms by UV spectroscopy” Pharm Analysis 2009 Vol 1 Issue 4 pp 351-353.
*Grinberg S, C. Linder, E. Heldman, Z. Weizman, and V. Kolot: EP1360168, 2003-11-12 and WO2002IL00043and 20020116, Filed by B G Negev “Amphiphilic Derivatives for the Production of Vesicles, Micelles, Complexants, and Uses Thereoff” in 2003
*Grinberg S., Linder C., Kolot V., Waner T., Wiesman Z., Shaubi E., Heldman E. (2005) Novel cationic amphiphilic derivatives from vernonia oil: synthesis and self-aggregation into bilayer vesicles, nanoparticles, and DNA complexants. Langmuir. 21(17):7638-7645.
*Grinberg S., Kolot V., Linder C., Shaubi E., Kas'yanov V., Deckelbaum R.J., Heldman E. (2008) Synthesis of novel cationic bolaamphiphiles from vernonia oil and their aggregated structures. Chem Phys Lipids 153(2):85-97.
*Grinberg, S., Kipnis, N., Linder, C., Kolot, V. and Heldman, E., (2010) Assymetric bolaamphiphiles from veronica oil designed for drug delivery. Eur. J. Lipid Sci. Technol., 112, 137-151.
*E. Heldman E, C. Linder, S. Grinberg Amphiphilic compounds and vesicles liposomes for organ-specified drug targeting” US patent Application 20060039962+WO03047499-2003-06-12
Highleyman, L (2009) HIV and the Brain BETA. 2009 Summer-Fall; 21(4):16-29.
*Hutter T, Linder C, Heldman E, Grinberg S (2011) Interfacial and self-assembly properties of bolaamphiphilic compounds derived from a multifunctional oil, Journal of Colloid and Interface Science, in press (doi: 10. 1016j. jcis. .08. 057).
Jonasdottir T J, Fisher D R, Borrebaek J, Bruland O S, Larsen R H (2006) First in vivo evaluation of liposome-encapsulated 223Ra as a potential alpha-particle-emitting cancer therapeutic agent. Anticancer Res. 26(4B):2841-2848.
Letendre S, Marquie-Beck J, Capparelli E, Best B, Clifford D, Collier A C, Gelman B B, McArthur J C, McCutchan J A, Morgello S, Simpson D, Grant I, Ellis R J; CHARTER Group. (2008) Validation of the CNS Penetration-Effectiveness rank for quantifying antiretroviral penetration into the central nervous system. Arch Neurol. 2008 January; 65(1):65-70.
*Linder C; Grinberg S; Heldman E “Nano-sized Particles Composing Multi-Headed Amphiphiles for Targeted Drug-Delivery” WO 2010128504 (A2) 2010.
Lu W, Tan Y Z, Hu K L and Jiang X G. (2005) Cationic albumin conjugated pegylated nanoparticle with its transcytosis ability and little toxicity against blood-brain barrier. Int J Pharm. May 13; 295 (1-2); 247-260.
New R. R. C. (ed). (1997) Liposomes. A Practical Approach. IRL Press, Oxford.
Newton H B (2006) Advances in strategies to improve drug delivery to brain tumors. Expert Rev Neurother. 6(10):1495-509.
*Popov M., Linder C., Deckelbaum R. J., Grinberg S., Hansen I. H., Shaubi E., Waner T., Heldman E. (2009) Cationic vesicles from novel bolaamphiphilic compounds. J Liposome Res. 20(2): 147-159.
*Popov M, Grinberg S, Linder C, Bachar Z, Waner T, Deckelbaum R, Heldman E. (2011) Site-directed decapsulation of bolaamphiphilic vesicles with enzymatic cleavable surface groups submitted to the Journal of Controlled Release.
*Puri, A., Loomis, K., Smith, B., Lee, J., Yavlovich, A., Heldman, E. and Blumenthal, R. (2009) Lipid-Based Nanoparticles as Pharmaceutical Drug Carriers: From Concepts to Clinic. Crit Rev Ther Drug Carrier Syst, 26(6): 523-580.
Saiyed Z, Gandhi N, and Nairi M (2010) Magnetic Nanoformulation of Azidothymidine 5′-triphosphate for Targeted Delivery across the BloodBrain Barrier. International Journal of Nanomedicine 5 :157-166
Songjiang Z and Lixiang W. (2009) Amyloid-Beta Associated with Chitosan Nano-Carrier has Favorable Immunogenicity and Permeates the BBB. AAPS Pharm Sci Tech, 10(3):900-905.
Spudich S and Antses B (2011) Central Nervous System Complications of HIV Infection. Top. Antiviral Med 19(2), 48-57.
Stern J, Freisleben H J, Janku S, Ring K. (1992) Black lipid membranes of tetraether lipids from Thermoplasma acidophilum, Biochim Biophys Acta 1128:227-236.
Varatharajan L and Thomas S. (2009)The transport of anti-HIV drugs across blood-CNS interfaces: Summary of current knowledge and recommendations for further Research Antiviral Res. 2009 May; 82(2): A99A109.
*Wiesman Z., Dom N. B., Sharvit E., Grinberg S., Linder C., Heldman E., Zaccai M. (2007) Novel cationic vesicle platform derived from vernonia oil for efficient delivery of DNA through plant cuticle membranes. J Biotechnol. 130(1):85-94.
*Zabicky J; Linder C; Grinberg S; Heldman E “Nano- and Mesosized Particles Comprising an Inorganic Core, Process and Applications Thereof” US2009011002

1. A pharmaceutical composition or a formulation comprising a bolaamphiphile complex, a sub-micron sized vesicle, or nano-sized vesicle; wherein the bolaamphiphile complex or nano-sized vesicles comprises one or more bolaamphiphilic compounds and a biologically active compound, wherein the bolaamphiphilic compound is a compound according to formula I:
HG²-L¹-HG¹ I
or a pharmaceutically acceptable salt, solvate, hydrate, prodrug, stereoisomer, tautomer, isotopic variant, or N-oxide thereof, or a combination thereof;
wherein:
each HG¹ and HG² is independently a hydrophilic head group; and
L¹ is alkylene, alkenyl, heteroalkylene, or heteroalkenyl linker; unsubstituted or substituted with C₁-C₂₀ alkyl, hydroxyl, or oxo.

2. A method of delivering biologically active compounds into animal or human brain comprising the step of administering to the animal or human a pharmaceutical composition or a formulation according to claim 1.

3-4. (canceled)

5. The pharmaceutical composition according to claim 1, wherein
L¹ is heteroalkylene, or heteroalkenyl linker comprising C, N, and O atoms; unsubstituted or substituted with C₁-C₂₀ alkyl, hydroxyl, or oxo.

6. (canceled)

7-9. (canceled)

10. The pharmaceutical composition according to claim 1, wherein the bolaamphiphilic compound is a compound according to formula II, III, IV, V, or VI: 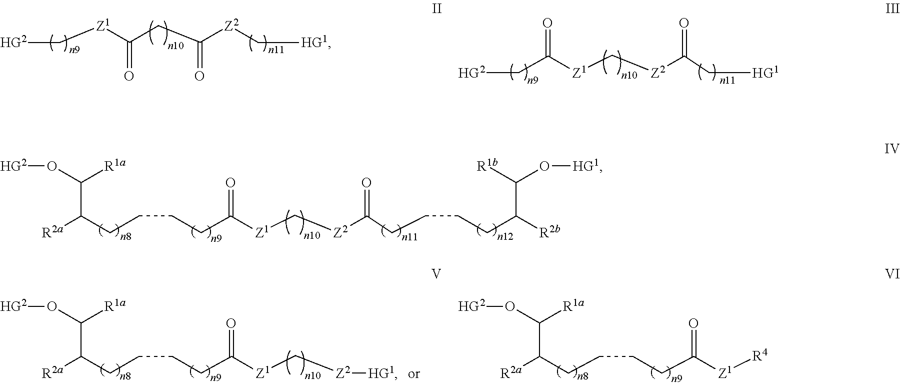
or a pharmaceutically acceptable salt, solvate, hydrate, prodrug, stereoisomer, tautomer, isotopic variant, or N-oxide thereof, or a combination thereof;
wherein:
each HG¹ and HG² is independently a hydrophilic head group;
each Z¹ and Z² is independently —C(R³)₂—, —N(R³)— or —O—;
each R^{1a}, R^{1b}, R³, and R⁴ is independently H or C₁-C₈ alkyl;
each R^{2a} and R^{2b} is independently H, C₁-C₈ alkyl, OH, alkoxy, or O—HG¹ or O—HG²;
each n8, n9, n11, and n12 is independently an integer from 1-20;
n10 is an integer from 2-20; and
each dotted bond is independently a single or a double bond.

11-13. (canceled)

14. The pharmaceutical composition according to claim 10, wherein the bolaamphiphilic compound is a compound according to formula II, III, IV, V, or VI; and each n8 and n12 is independently 1, 2, 3, or 4.

15. (canceled)

16. The pharmaceutical composition according to claim 10, wherein the bolaamphiphilic compound is a compound according to formula II, III, IV, V, or VI; and each R^{2a} and R^{2b} is independently H, OH, alkoxy, or O—HG¹ or O—HG².

17-18. (canceled)

19. The pharmaceutical composition according to claim 10, wherein the bolaamphiphilic compound is a compound according to formula II, III, IV, V, or VI; and each R^{1a} and R^{1b} is independently H, Me, Et, n-Pr, i-Pr, n-Bu, i-Bu, sec-Bu, n-pentyl, isopentyl, n-hexyl, n-heptyl, or n-octyl.

20-22. (canceled)

23. The pharmaceutical composition according to claim 10, wherein the bolaamphiphilic compound is a compound according to formula II, III, IV, or V; n10 is an integer from 2-16.

24-25. (canceled)

26. The pharmaceutical composition according to claim 10, wherein the bolaamphiphilic compound is a compound according to formula VI; and R⁴ is H, Me, Et, n-Pr, i-Pr, n-Bu, i-Bu, sec-Bu, n-pentyl, or isopentyl.

27-28. (canceled)

29. The pharmaceutical composition according to claim 10, wherein the bolaamphiphilic compound is a compound according to formula II, III, IV, V, or VI; and each Z¹ and Z² is independently C(R³)₂—, or —N(R³)—; and each R³ is independently H, Me, Et, n-Pr, i-Pr, n-Bu, i-Bu, sec-Bu, n-pentyl, or isopentyl.

30. (canceled)

31. The pharmaceutical composition according to claim 10, wherein the bolaamphiphilic compound is a compound according to formula II, III, IV, V, or VI; and each Z¹ and Z² is —O—.

32. The pharmaceutical composition according to claim 1, wherein the bolaamphiphilic compound is a compound according to formula II, III, IV, V, or VI; and each HG¹ and HG² is independently selected from: 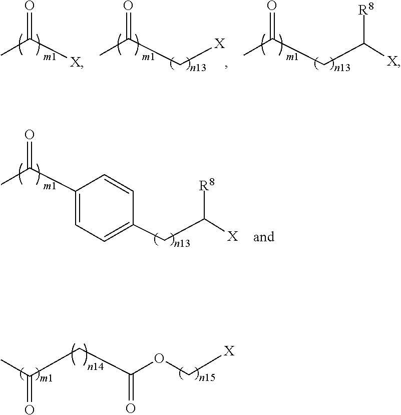
wherein:
X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R⁵′ is independently substituted or unsubstituted C₁-C₂₀ alkyl;
each R⁸ is independently H, substituted or unsubstituted C₁-C₂₀ alkyl, alkoxy, or carboxy;
m1 is 0 or 1; and
each n13, n14, and n15 is independently an integer from 1-20.

33-37. (canceled)

38. The pharmaceutical composition according to claim 1, wherein the bolaamphiphilic compound is a compound according to formula VIIa, VIIb, VIIc, or VIId: 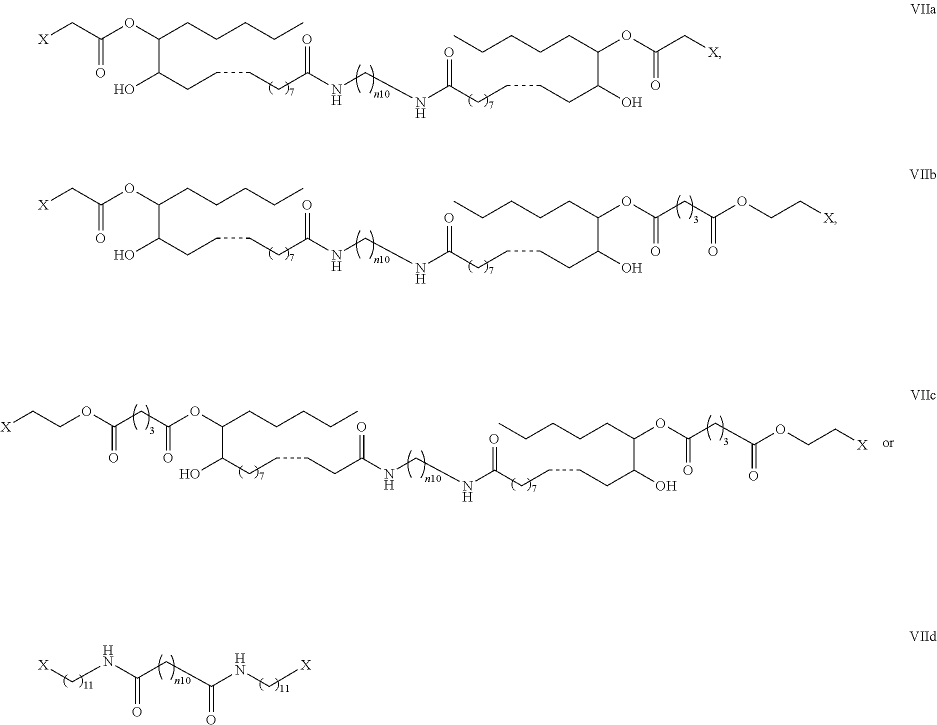
or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl;
n10 is an integer from 2-20; and
each dotted bond is independently a single or a double bond.

39. The pharmaceutical composition according to claim 1, wherein the bolaamphiphilic compound is a compound according to formula VIIIa, VIIIb, VIIIc, or VIIId: 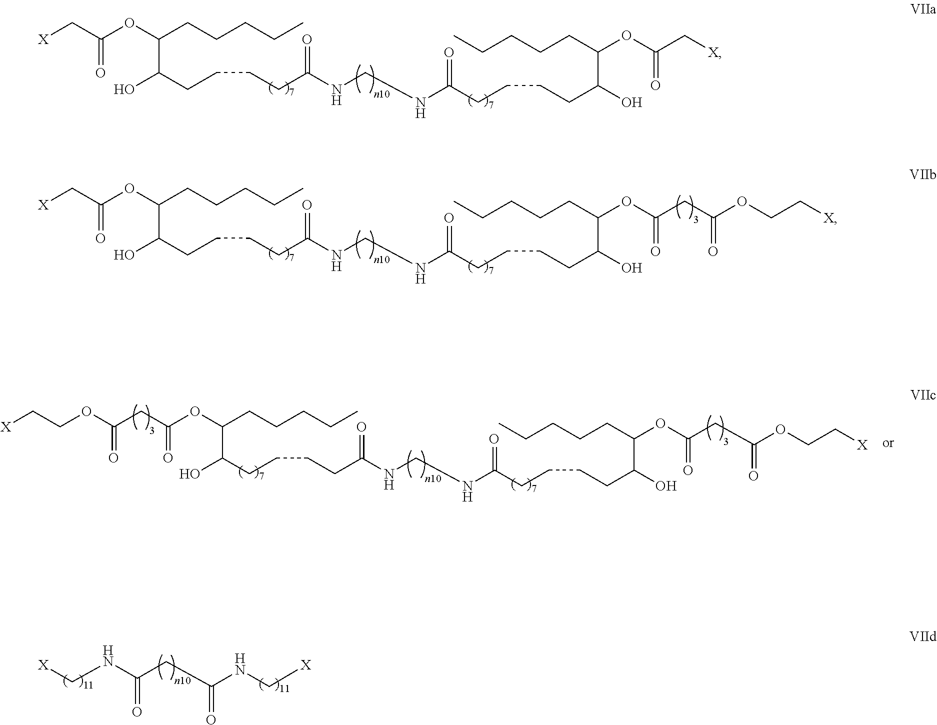
or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl;
n10 is an integer from 2-20; and
each dotted bond is independently a single or a double bond.

40. The pharmaceutical composition according to claim 1, wherein the bolaamphiphilic compound is a compound according to formula IXa, IXb, or IXc: 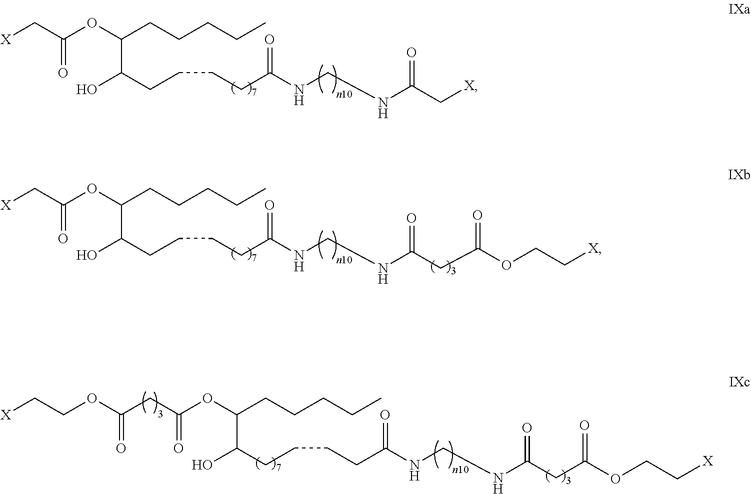
or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl;
n10 is an integer from 2-20; and
each dotted bond is independently a single or a double bond.

41. The pharmaceutical composition according to claim 1, wherein the bolaamphiphilic compound is a compound according to formula Xa, Xb, or Xc: 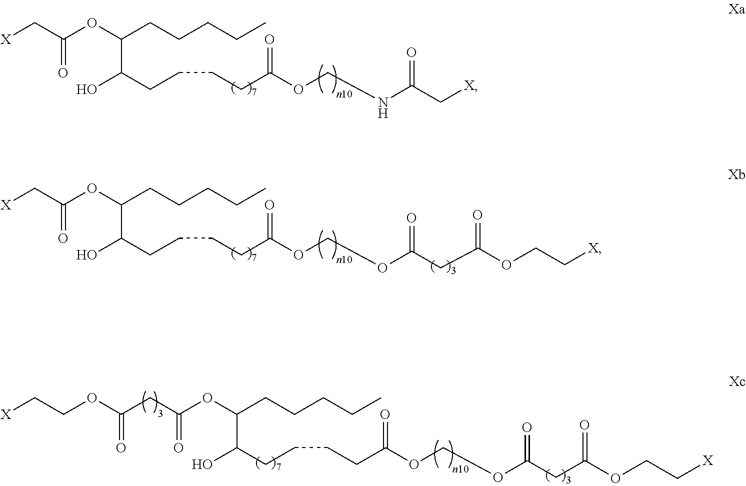
or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl;
n10 is an integer from 2-20; and
each dotted bond is independently a single or a double bond.

42-43. (canceled)

44. The pharmaceutical composition according to claim 38, wherein the bolaamphiphilic compound is a compound according to formula VIIa-VIId, VIIIa-VIIId, IXa-IXc, or Xa-Xc; n10 is an integer from 2-16.

45-46. (canceled)

47. The pharmaceutical composition according to claim 32, wherein each R^{5a}, R^{5b}, and R^{5c} is independently substituted or unsubstituted C₁-C₂₀ alkyl.

48-49. (canceled)

50. The pharmaceutical composition according to claim 32, wherein two of R^{5a}, R^{5b}, and R^{5c} are independently C₁-C₂₀ alkyl substituted with —OC(O)R⁶; and R⁶ is C₁-C₂₀ alkyl.

51. The pharmaceutical composition according to claim 32, wherein one of R^{5a}, R^{5b}, and R^{5c} is C₁-C₂₀ alkyl substituted with —OC(O)R⁶; and R⁶ is Me, Et, n-Pr, i-Pr, n-Bu, i-Bu, sec-Bu, n-pentyl, isopentyl, n-hexyl, n-heptyl, or n-octyl.

52. The pharmaceutical composition according to claim 32, wherein one of R^{5a}, R^{5b}, and R^{5c} is C₁-C₂₀ alkyl substituted with amino, alkylamino or dialkylamino.

53. (canceled)

54. The pharmaceutical composition according to claim 32, wherein R^{5a}, and R^{5b} together with the N they are attached to form substituted or unsubstituted heteroaryl.

55. (canceled)

56. The pharmaceutical composition according to claim 32, wherein R^{5a}, and R^{5b} together with the N they are attached to form substituted or unsubstituted monocyclic or bicyclic heterocycle.

57-60. (canceled)

61. The pharmaceutical composition according to claim 32, wherein X is —N(Me)-CH₂CH₂—OAc or —N⁺(Me)₂-CH₂CH₂—OAc.

62. The pharmaceutical composition according to claim 32, wherein X is a chitosanyl group.

63. The pharmaceutical composition according to claim 32, wherein X is a substance P head group.

64. The pharmaceutical composition according to claim 32, wherein X is a substance P head group, and the substance P head group is bound through the ω-amino group of lysine.

65. The pharmaceutical composition according to claim 32, wherein X is —NH—(CH₂)₄—C(H)(NH-Pro-Arg)-NH-Pro-Gly-Gly-Phe-Phe-Gly-Leu-Met.

66. The pharmaceutical composition according to claim 32, wherein X is a headgroup comprising NK1R antagonist.

67. The pharmaceutical composition according to claim 32, wherein X is a headgroup comprising NK1R antagonist, and the NK1R antagonist is I, II, or III:

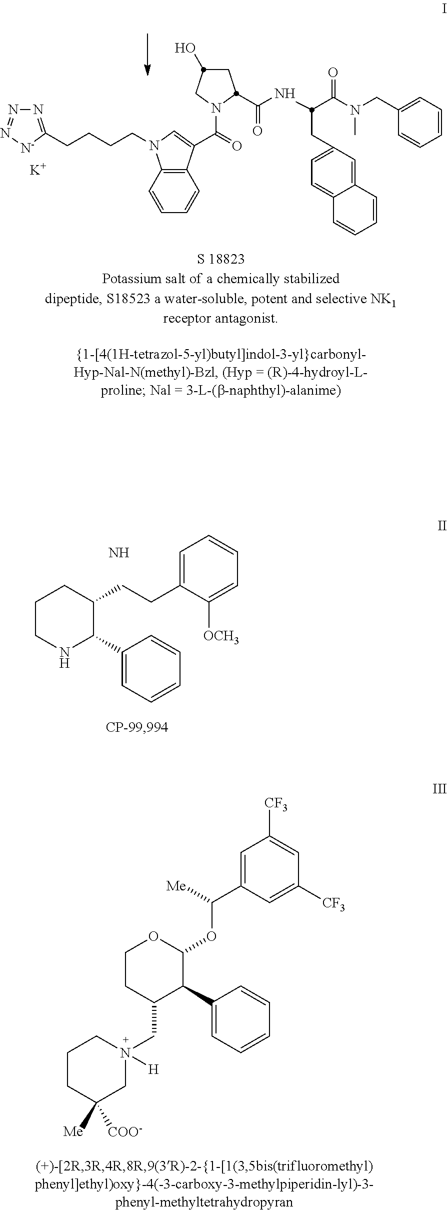

68. The pharmaceutical composition according to claim 1, wherein the bolaamphiphilic compound is a pharmaceutically acceptable salt.

69. The pharmaceutical composition according to claim 1, wherein the bolaamphiphilic compound is in a form of a quaternary salt.

70. (canceled)

71. The pharmaceutical composition according to claim 1, wherein the bolaamphiphilic compound is any one of the bolaamphiphilic compounds listed in Table 1.

72-80. (canceled)

81. The pharmaceutical composition of claim 1, wherein the biologically active compound is an analgesic.

82. The pharmaceutical composition of claim 1, wherein the biologically active compound is a drug active against ALS.

83. The pharmaceutical composition of claim 1, wherein the biologically active compound is a drug active against brain tumor.

84. The pharmaceutical composition of claim 1, wherein the biologically active compound is kyotorphine or enkephaline.

85. The pharmaceutical composition of claim 1, wherein the biologically active compound is CPT-11, or BCNU (Carmustine).

86-88. (canceled)

89. The pharmaceutical composition of claim 1, wherein the biologically active compound is a cyclodextrin, a calcium channel antagonist, or a calcium channel agonist.

90. The pharmaceutical composition of claim 89, wherein the cyclodextrin encapsulates at least one biologically-active molecule.

91. The pharmaceutical composition of claim 90, wherein the biologically-active molecule is a peptide, protein, or nucleic acid.

92. A method for treatment of a disease or condition comprising administration of a therapeutically-effective amount of a pharmaceutical composition of claim 1 to a patient in need thereof, wherein the disease or condition is Parkinson's disease, Alzheimer's disease, lysosomal storage disease, brain tumor, or pain.

93. The method of claim 92, wherein the lysosomal storage disease or condition is Niemann-Pick disease.

94. The method of claim 93, wherein the biologically-active agent is cyclodextrin.

95. The method of claim 92, wherein the brain tumor is glioblastoma multiforme.

96. The method of claim 95, wherein the biologically-active compound is CPT-11.

97. The method of claim 95, wherein the pharmaceutical composition is a pharmaceutical composition according to claim 63.

98. The method of claim 95, wherein the pharmaceutical composition is a pharmaceutical composition according to claim 64.

99. The method of claim 95, wherein the pharmaceutical composition is a pharmaceutical composition according to claim 65.

100. The method of claim 95, wherein the pharmaceutical composition is a pharmaceutical composition according to claim 66.

101. The method of claim 95, wherein the pharmaceutical composition is a pharmaceutical composition according to claim 67.

102. The method of claim 92, wherein the disease or condition is Parkinson's disease or Alzheimer's disease and the biologically-active agent is a neurotrophic factor.

103. The method of claim 102, wherein the neurotrophic factor is glial cell-derived neurotrophic factor (GDNF).
